(19) United States
(12) Patent Application Publication
(10) Pub. No.: US 2002/0102577 A1
(43) Pub. Date: Aug. 1, 2002

(54) Nucleotide incorporating enzymes

(76) Inventors: Sun Ai Raillard, Mountain View, CA (US); Mark Welch, Fremont, CA (US); Jon Ness, Sunnyvale, CA (US)

Correspondence Address: LAW OFFICES OF JONATHAN ALAN QUINE, P O BOX 458, ALAMEDA, CA, 94501

(21) Appl. No.: 09/920,452

(22) Filed: Jul. 31, 2001

Related U.S. Application Data
(60) Provisional application No. 60/244,764, filed on Oct. 31, 2000.
(60) Provisional application No. 60/222,056, filed on Jul. 31, 2000.

(51) Int. Cl. C12Q 1/68; G06F 19/00; G01N 33/48; G01N 33/50; C12P 19/34; C12N 9/22

(52) U.S. Cl. 435/6; 435/91.2; 435/199; 702/20

(57) ABSTRACT

Nucleotide incorporating enzyme variants having improved properties including the ability to utilize non-natural or rare nucleotide analogues in the synthesis of polynucleotides are provided. Methods for producing such nucleotide incorporating enzyme variants are provided, as are kits and integrated systems including the nucleotide incorporating enzyme variants.

### CROSS-REFERENCE TO RELATED APPLICATIONS

This application claims priority to and benefit of U.S. Provisional Applications No. 60/244,764, filed Oct. 31, 2000, and No. 60/222,056, filed Jul. 31, 2000, the disclosures of each of which are incorporated herein in their entirety for all purposes.

### COPYRIGHT NOTIFICATION PURSUANT TO 37 C.F.R. §1.71(e)

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

Numerous applications utilizing DNA and RNA polymerases, and other enzymes capable of catalyzing the formation of a phosphodiester bond between adjacent nucleotides in a polynucleotide, are used in the experimental and therapeutic manipulation of polynucleotides. To optimize performance of many of these reactions, polymerases with specialized properties, such as thermostability, reduced exonuclease activity, and the like, are desirable. Typically, access to such enzymes is dependent either on the isolation of an enzyme with the desired property from a natural source or by site-specific mutagenesis. Both approaches require large expenditures, either in finding the natural source (e.g., a thermophilic bacteria) or in determining the three-dimensional structure and relevant functional groups important for the desired characteristic.

The present invention provides enzyme variants that efficiently incorporate non-natural and/or rare nucleotide analogues, as well as enzyme variants with additional or alternative beneficial properties. Methods for producing such enzyme variants are also provided. Additional benefits will become apparent from review of the detailed description of the invention.

### SUMMARY OF THE INVENTION

The present invention provides nucleotide incorporating enzyme variants (e.g., nucleic acid polymerases, nucleotidyl terminal transferases, ligases, reverse transcriptases, RNA polymerases, and telomerases) with novel and desirable properties, and methods for their production. One aspect of the invention relates to nucleotide incorporating enzymes that efficiently incorporate non-natural and rare nucleotide analogues. In some embodiments, the methods of the invention involve identifying a non-natural or rare nucleotide analogue of interest, typically such a nucleotide analogue is incorporated into an elongating polynucleotide by existing nucleotide incorporating enzymes at an efficiency of less than 10% the efficiency at which a naturally occurring nucleotide, e.g., A, C, G, T or U, is incorporated by the same enzyme. Nucleic acid segments encoding all or part of a parental polymerase and/or other nucleotide incorporating enzyme are diversified using any of a variety of diversity generating procedures to produce a library of nucleic acids encoding nucleotide incorporating enzyme variants. The library is then evaluated by one or more selection or screening methods to identify at least one nucleotide incorporating enzyme variant that efficiently incorporates the non-natural or rare nucleotide analogue.

In some embodiments, a non-natural or rare nucleotide analogue that is incorporated with an efficiency of less than 10% the efficiency of a naturally occurring nucleotide is identified. In other embodiments, non-natural or rare nucleotide analogues that are incorporated at less than about, e.g., 5%, 1%, 0.05%, or 0.01%, or less, the rate of a naturally occurring nucleotide are identified. For example, nucleotide analogues selected from among nucleotides derivatized with functional groups (such as methyl and other alkyl, nitrile, formyl, carbonyl, carboxy, halogen, nitroso, or aryl groups), nucleotides comprising unnatural base analogues, nucleotides comprising fluorescent labels, nucleotides comprising ribose or deoxyribose analogues, nucleotides comprising unnatural glycosidic linkages, and nucleotides comprising unnatural backbone chemistry are identified in various embodiments of the invention. In certain embodiments, non-natural or rare nucleotide analogues that are incorporated less efficiently than inosine, xanthine or 7-deaza dGTP are identified.

Regardless of the nucleotide analogue identified, the methods of the invention are optionally employed to produce a nucleotide incorporating enzyme variant that incorporates the identified nucleotide analogue with increased efficiency. In some embodiments, the nucleotide incorporating enzyme variant incorporates the non-natural or rare nucleotide analogue at least about 10% as efficiently as it incorporates a naturally occurring nucleotide. In other embodiments, the nucleotide analogue is incorporated at least about 10 fold more efficiently than the one or more parental nucleotide incorporating enzyme from which it is derived. Alternatively, the nucleotide incorporating enzyme incorporates the identified non-natural or rare nucleotide analogue at least about 20 fold, 50 fold, or 100 fold more efficiently than a parental nucleotide incorporating enzyme.

Another aspect of the invention relates to nucleotide incorporating enzymes with improved nucleotide incorporating activity under a variety of reaction conditions. In some embodiments, the nucleotide incorporating enzymes maintain activity in the presence of contaminants, such as impurities found in such biological fluid samples as blood, plasma and urine. In some embodiments, the nucleotide incorporating enzyme variants are DNA polymerases, such as RNA dependent DNA polymerases, reverse transcriptases, or RNA polymerases.

In certain embodiments, the nucleotide incorporating enzymes, such as the reverse transcriptases described above, are thermostable, maintaining efficient activity under conditions suitable for amplification of polynucleotide templates, such as polymerase chain reaction conditions. In some embodiments, a thermostable DNA polymerase of the invention incorporates dUTP with high efficiency.

The methods of the invention involve diversification of a plurality of nucleic acid segments encoding all or part of one or more parental nucleotide incorporating enzymes to produce a library of nucleic acids encoding nucleotide incorporating enzyme variants. Nucleotide incorporating enzymes include DNA polymerases, RNA polymerases, terminal transferases, ligases, telomerases, and the like. The nucleic acid segments are DNA or RNA polynucleotides, or alternatively, character strings representing DNA or RNA polynucleotides stored in a computer readable medium. In the case of physical embodiments of a polynucleotide sequence, segments are produced by any one or more of enzymatic digestion, chemical cleavage, mechanical fragmentation or artificial synthesis. In certain embodiments, the parental nucleotide incorporating enzymes are derived from one or more thermophilic organisms, such as a thermophilic bacteria, e.g., of a Thermus species.

In one embodiment, the nucleic acid segments encode inactive nucleotide incorporating enzymes or homologues thereof. In another embodiment, the nucleic acid segments include codon altered nucleic acid segments. In another embodiment, the nucleic acid segments include at least one nucleic acid segment with introns or inteins. In yet another embodiment, the nucleic acid segments include two or more members of a family of nucleotide incorporating enzymes. While in yet another embodiment, the nucleic acid segments include two or more families of nucleotide incorporating enzymes.

The nucleic acid segments are subjected to at least one diversity generating procedure involving recombining or mutating the nucleic acid segments. In some embodiments, the nucleic acid segments are recombined or recursively recombined in vitro, in vivo, or in silico. In an embodiment, synthetic oligonucleotides are assembled to produce a library of nucleic acids encoding nucleotide incorporating enzyme variants.

In other embodiments, the nucleic acid segments are diversified by error prone PCR, and the amplified product is optionally recombined. In another embodiment, at least one nucleic acid segment is mutated to produce one or more mutated nucleic acid segments, which are optionally recombined.

In some embodiments, the nucleic acids encoding nucleotide incorporating enzyme variants also include a vector, for example, a replicable vector.

The nucleotide incorporating enzyme variants are evaluated by any of a variety of screening or selection procedures to identify variants that efficiently incorporate the non-natural or rare nucleotide analogue of interest. In one embodiment, the nucleotide incorporating enzyme variant is identified by mass spectroscopy. In other embodiments, nucleotide incorporating enzyme variants are identified by optical or fluorescent spectroscopy, radiometry, chromatography, gel electrophoresis, capillary electrophoresis, avidin (or streptavidin) binding, hybridization, fluorescent resonance energy transfer, fluorescent polarization or pyrophosphate detection. In some embodiments, the nucleotide incorporating enzyme variants are identified in a high throughput assay format. In some embodiments, the library of nucleic acids encoding nucleotide incorporating enzyme variants are screened or pre-screened in cellular complementation assays.

In some embodiments, nucleotide incorporating enzyme variants are identified that possess one or more additional desired property as well as incorporating a specified non-natural or rare nucleotide analogue. For example, nucleotide incorporating enzymes with desired properties such as thermostability, evenness of nucleotide incorporation, efficient terminal transferase activity, low fidelity, high fidelity, processivity, strand-displacement activity, nick translation activity, exchange reaction, cation requirement, modulation of activity by cation, sulfhydral reagent requirement, shelf life, salt tolerance, organic solvent tolerance, mechanical stress tolerance, tolerance to impurities, altered pH dependence, altered dependence on buffer conditions, template composition, primer composition, and improved stability are identified according to the methods of the invention. In one embodiment, such nucleotide incorporating enzyme variants are identified by simultaneously screening for incorporation of the non-natural or rare nucleotide analogue and at least one other desired property.

Another aspect of the invention relates to the use of nucleotide incorporating enzymes produced according to the methods of the invention in polymerase chain reactions (e.g., to yield balanced PCR products or to be able to disrupt secondary structures by elevated temperature stability during the amplification reaction), sequencing reactions or other primer extension reactions in vitro.

Nucleotide incorporating enzyme variants that efficiently incorporate non-natural or rare nucleotide analogues are a feature of the invention. Such enzyme variants are produced according to the methods of the invention. In one embodiment, the nucleotide incorporating enzyme variant incorporates the non-natural or rare nucleotide analogue of interest at least about 10% as efficiently as a naturally occurring nucleotide. In another embodiment, the nucleotide incorporating enzyme variant incorporates the non-natural or rare nucleotide analogue at least about 10 fold, 20 fold, 50 fold or 100 fold more efficiently than a parental nucleotide incorporating enzyme. In one embodiment, the nucleotide incorporating enzyme variants incorporate nucleotides and/or nucleotide analogues with low fidelity. In an alternative embodiment, the nucleotide incorporating enzyme variants incorporate nucleotides and/or nucleotide analogues with high fidelity.

In yet other embodiments, in the method for producing a nucleotide incorporating enzyme that incorporates a non-natural or rare nucleotide analogue, diversification comprises: generating a plurality of partially duplexed oligonucleotides by hybridizing the oligonucleotides (e.g., nucleic acid segments encoding all or part of one or more parental nucleotide incorporating enzymes or homologues thereof), which duplexed oligonucleotides have overhangs of unhybridized regions; assembling the plurality of partially duplexed oligonucleotides by hybridizing the overhangs of two or more partially duplexed oligonucleotides together; and ligating the assembled oligonucleotides to produce a library of recombinant nucleic acids, optionally in the presence of a polymerase.

In some embodiments, in the method for producing a nucleotide incorporating enzyme with increased tolerance to biological impurities, diversification comprises: generating a plurality of partially duplexed oligonucleotides by hybridizing the oligonucleotides (e.g., nucleic acid segments encoding all or part of one or more parental nucleotide incorporating enzymes or homologues thereof), said duplexed oligonucleotides having overhangs of unhybridized regions; assembling the plurality of partially duplexed oligonucleotides by hybridizing the overhangs of two or more partially duplexed oligonucleotides together; and ligating the assembled oligonucleotides to produce a library of recombinant nucleic acids (optionally in the presence of a polymerase).

In some aspects, the invention comprises a method for identifying a nucleotide incorporating enzyme having a desired property, the method comprising: providing a plurality of partially duplexed oligonucleotides having overhangs of unhybridized regions, which oligonucleotides comprise a subsequence of a nucleic acid encoding a nucleotide incorporating enzyme; assembling the plurality of partially duplexed oligonucleotides by hybridizing the overhangs of two or more partially duplexed oligonucleotides together; ligating the assembled oligonucleotides to produce a library of recombinant nucleic acids (optionally in the presence of a polymerase); expressing the recombinant nucleic acids to generate a library of nucleotide incorporating enzyme variants; and, screening the library of nucleotide incorporating enzyme variants for one or more desired property (e.g., a desired property selected from: thermostability, evenness of nucleotide incorporation, efficient terminal transferase activity, low fidelity, high fidelity, processivity, strand-displacement activity, nick translation activity, exchange reaction, cation requirement, modulation of activity by cation, sulfhydral reagent requirement, shelf life, salt tolerance, organic solvent tolerance, mechanical stress tolerance, tolerance to impurities, altered pH dependence, altered dependence on buffer conditions, template composition, primer composition, and improved stability).

Kits including a nucleotide incorporating enzyme variant of the invention, and one or more of a container, a packaging material, and a non-natural or rare nucleotide analogue are also a feature of the invention. Integrated systems comprising a non-natural nucleotide analogue, a nucleotide incorporating enzyme variant of the invention, and a detector are also a feature of the invention. Optionally such integrated systems include one or more of a user input device, a data processing device, a data output device, and/or a robotic controller.

### BRIEF DESCRIPTION OF THE FIGURES

FIG. 1 illustrates the phylogenetic relationship between multiple Thermus DNA polymerases.

FIG. 2 schematically illustrates the detection of an amplified target sequence using a molecular beacon.

FIG. 3 schematically illustrates capture and detection of a target nucleic acid using a reporter.

FIG. 4 schematically illustrates a High Throughput solid phase screen for polymerase activity.

### DETAILED DESCRIPTION OF THE INVENTION

The use of nucleic acid polymerases could be widely extended if non-natural nucleotide analogues, such as fluorescently labeled nucleotides, nucleotides derivatized with functional groups (such as methyl and other alkyl, nitrile, formyl, carbonyl, carboxy, halogen, nitroso, or aryl groups), nucleotides comprising unnatural base analogues, nucleotides comprising ribose or deoxyribose analogues, nucleotides comprising unnatural glycosidic linkages, and nucleotides with unnatural backbone chemistry such as phosphonate, amide, and tholate, could be efficiently incorporated into the polynucleotide synthesized.

For example, nucleic acid polymerases that efficiently incorporate fluorescently labeled nucleotides would facilitate detection of polynucleotides in a variety of settings, including sequencing of nucleic acids, detection of amplified products, e.g., by PCR, labeling of probes, etc. A nucleotide analogue in its triphosphate form (wherein the sugar is ribose, deoxyribose, or dideoxyribose) can be labeled either at the base or at the sugar moiety, (e.g., in the 2′ or 3′ hydroxyl position). Typically, the fluorescent label, e.g., fluorescein, is coupled via a linker.

Matrix-assisted laser desorption/ionization (MALDI) mass spectrometry (MS) is potentially the next generation of sequencing tool, replacing electrophoretic separation of the extended strand in the Sanger dideoxy termination sequencing method (Smith et al., (1996) Nat Biotech 14:1084). A significant limitation of MALDI-MS sequencing is the difficulty of analyzing intact long polynucleotides. To date, the longest single-stranded oligonucleotide reported to have been analyzed is only 89 nucleotides in length (Wu et al. (1994) Anal Chem 66:1637-1645). The single most important reason for this limitation is the fragmentation of DNA molecules consisting of naturally occurring nucleotides under MALDI conditions. However, this problem can be ameliorated by using non-natural nucleotide analogues that are known to be more stable. Unfortunately, such non-natural nucleotide analogues are poorly incorporated by existing DNA polymerases.

The present invention provides nucleotide incorporating enzyme variants, for example, DNA and RNA polymerases, nucleotidyl terminal transferases, ligases, telomerases, etc. that are capable of enzymatically incorporating non-natural or rare nucleotides into an elongating polynucleotide. Such enzymes are of significant utility in a wide range of research and commercial applications. For example, a DNA polymerase that efficiently incorporates fluorescently labeled nucleotide analogues is valuable for, among other applications, sequencing, fingerprinting and single nucleotide polymorphism (SNP) analysis.

The invention also provides methods for producing nucleotide incorporating enzyme variants, e.g., DNA polymerases, RNA polymerases, terminal transferases, etc., that incorporate non-natural and/or rare nucleotide analogues. In a general aspect, the methods of the invention relate to the diversification of nucleic acids corresponding to, i.e., encoding, one or more nucleotide incorporating enzyme, or homologue thereof, to produce a library of recombinant nucleic acids encoding nucleotide incorporating enzyme variants, members of which library can incorporate a non-natural or rare nucleotide analogue into an extending polynucleotide.

More specifically, the methods of the invention involve the identification of a non-natural or rare nucleotide analogue that is only poorly incorporated into a polynucleotide by an existing polymerase, or group of polymerases. Typically, the non-natural or rare nucleotide analogue is incorporated into a polynucleotide with an efficiency of less than about 10%, or less than about 5%, about 1%, about 0.05%, about 0.01%, about 0.005%, about 0.001%, or even less, than the efficiency at which a naturally occurring nucleotide is incorporated. Nucleic acid segments, i.e., DNA or RNA polynucleotides, or character strings representing DNA or RNA polynucleotides, encoding all or part of, one or more than one, nucleotide incorporating enzyme are then recombined and/or mutated to generate a diverse library of nucleic acids that encode nucleotide incorporating enzyme variants. A variety of in vivo and in vitro screening and selection assays are then employed to identify variants that incorporate a specified non-natural or rare nucleotide analogue with increased efficiency relative to one or more parental nucleotide incorporating enzymes.

Definitions

Unless defined otherwise, all scientific and technical terms are understood to have the same meaning as commonly used in the art to which they pertain. For the purpose of the present invention, the following terms are defined below.

The term “nucleotide incorporating enzyme” is used herein to refer to any enzyme, enzyme group, or enzyme class, that in a template dependent or independent manner, covalently incorporates one or more nucleotides (or nucleotide analogues) into an oligonucleotide or polynucleotide. Such incorporated nucleotides optionally can be in the form of, e.g., dinucleotides, trinucleotides, etc., or in larger groupings of nucleotides such as nucleotide lengths comprising at least 4, 5, 6, 7, 8, 9, 10, 15, 20, 25, 50, 75, 100, or 500 or more nucleotides. The nucleotides incorporated by a nucleotide incorporating enzymes of the invention optionally can comprise natural nucleotides, nucleotide analogues, non-natural nucleotide analogues, rare nucleotides, or any combination of the above. Examples of nucleotide incorporating enzymes include numerous classes of DNA polymerases, including reverse transcriptases, RNA polymerases, terminal transferases, DNA ligases, and telomerases.

The term “nucleic acid polymerase,” generically, refers to any of a subset of nucleotide incorporating enzyme that, in a template dependent manner, elongates at least one strand of nucleotides, e.g., a polynucleotide, by sequentially incorporating single nucleotides, typically, in a 5′ to 3′ direction. Nucleic acid polymerases include both DNA (DNA dependent DNA polymerases; RNA dependent DNA polymerases or reverse transcriptases) and RNA polymerases (DNA dependent RNA polymerases; RNA dependent RNA polymerases).

The term “non-natural nucleotide analogue” (also described herein as “unnatural nucleotide,” “unnatural base analogue,” etc.) is used to designate a nucleotide analogue that is not found in nature, i.e., is man-made, artificial or synthetically derived. For example, nucleotides with modified bases or sugar moieties, e.g., nucleotides labeled at the 2′ or 3′ hydroxyl with a fluorescent tag, regardless of whether they are produced enzymatically or chemically, in vivo or in vitro, are considered non-natural nucleotide analogues. For the purpose of the present application a “rare” nucleotide, or nucleotide analogue, is an nucleotide analogue, other than adenosine, guanosine, cytidine, thymidine or uridine that is not typically added by a nucleotide incorporating enzyme during the synthesis of a polynucleotide.

The term “nucleic acid segment” refers to a contiguous length of nucleotides, e.g., a fragment of a polynucleotide, an oligonucleotide, whether embodied in physical form, e.g., a DNA or RNA polynucleotide or oligonucleotide, or represented by a character string in a computer readable medium.

A polynucleotide (oligonucleotide) is said to “encode” a polypeptide (oligopeptide) when the information inherent in a contiguous sequence of nucleotides can be translated, physically or logically, into a corresponding sequence of amino acids according to well-known rules, e.g., as set forth in Table 2, below. The sequence of amino acids can be contiguous or non-contiguous. For example, when the sequence of nucleotides translates into one or more “stop” codons, the polynucleotide “encodes” non-contiguous polypeptide (or peptide) fragments. It should be noted that these fragments are optionally rejoined through, e.g, intein mediated recombination.

A “codon altered” nucleic acid segment is a nucleic acid segment in which one or more nucleotides has been altered without altering the amino acids encoded by the sequence. For example, alterations in the third position of many codons, see, e.g., Table 2, do not result in changes in the amino acid encoded due to the redundancy of the genetic code. The use of codon altered nucleic acids can be particularly desirable when optimizing expression for a specific host cell type as codon usage is known to vary between organisms, i.e., especially between organisms of different phyla and kingdoms.

The term “parental” when referring to a polynucleotide or a polypeptide, such as an enzyme, e.g., a nucleotide incorporating enzyme or a nucleic acid polymerase, indicates a reference polynucleotide or polypeptide from which a set of variants are derived by one or more diversity generating procedure, e.g., recombination and/or mutation. The parental polynucleotide or polypeptide can be naturally occurring, i.e., identical to a sequence found in nature, or can itself be produced by one or more mutagenesis or recombination procedure or in in silico design.

The “efficiency” of incorporation of a nucleotide analogue refers to the relative rate at which it is incorporated into a polynucleotide by a nucleotide incorporating enzyme, as compared to the rate at which a reference nucleotide is incorporated by the same enzyme. Typically, the relative rate of incorporation, and thus, the efficiency is determined by comparison to a naturally occurring nucleotide, such as adenosine, guanosine, cytidine, thymidine or uridine.

A “labeled” nucleotide analogue, is any nucleotide or nucleotide analogue that is covalently joined to a detectable chemical moiety. The detectable chemical moiety can be detected indirectly, e.g., biotin, or directly, e.g., fluorescent labels such as fluorescein, rhodamine, and the like.

An “artificially synthesized” nucleic acid segment is a segment of a DNA or RNA (or DNA/RNA) molecule that is chemically or enzymatically produced ex vivo.

The term “family” when referring to a group of polypeptides (e.g., enzymes) or polynucleotides (e.g., encoding enzymes) indicates that the polypeptides or polynucleotides are related in structure (especially primary structure) and function. Typically, members of a family, e.g., a family of DNA polymerases, are more closely related to each other in sequence and structure, than to other families of polypeptides or polynucleotides. When members of a family of polypeptides or polynucleotides are related by descent from a common ancestral sequence, they are considered to be “homologues.”

A “vector” is used in connection with a nucleic acid as a means for introducing an exogenous nucleic acid into a cell, or for moving a nucleic acid from one cell to another cell. A vector can include naked nucleic acids, conjugated nucleic acids, liposomes, viruses, plasmids, phage, phagemids, cosmids, artificial chromosomes, etc. A vector capable of autonomous reproduction in a cell, e.g, a virus, phage, plasmid, cosmid, artificial chromosome, or the like, is designated a “replicable vector.” Vectors are optionally designed to localize a protein product to a particular cell compartment such as the cytoplasm or periplasm of, e.g., Escherichia coli. Vectors are also optionally designed to fuse the protein to a surface protein of a cell, virus, or phage, etc.

Nucleotide Incorporating Enzymes

To simplify discussion of the present invention, the term “nucleotide incorporating enzyme” is used to refer to any of several classes of enzymes with several distinct activities. The groups of enzymes share the common characteristic that they promote the addition of one or more nucleotides to an existing polynucleotide or oligonucleotide, e.g., a primer, by catalyzing the reaction of a 3′ hydroxyl on the polynucleotide or oligonucleotide with a 5′ phosphate group on a free nucleotide, or group of nucleotides. The enzymes can be divided into functional groupings based on their substrate and template requirements. Further sub-divisions can be made on the basis of structural and sequence similarities, primarily stemming from homology relationships between enzymes, and the nucleic acids encoding them. Sequences selected from among any one or more of the enzyme classes, from either prokaryotic or eukaryotic sources, are suitable substrates for diversification and selection according to the methods of the invention to produce a nucleotide incorporating enzyme capable of incorporating non-natural or rare nucleotide analogues into a polynucleotide.

Nucleic Acid Polymerases

The largest and most diverse category of nucleotide incorporating enzymes are the nucleic acid polymerases. Nucleic acid polymerases, i.e., DNA and RNA polymerases, are enzymes that catalyze the template directed synthesis of DNA and RNA molecules. A variety of polymerases have been isolated from various organisms, and many have proven useful for the in vitro synthesis of nucleotide macromolecules. Nucleic acid polymerases can be divided into four major categories: DNA-dependent DNA polymerases (EC 2.7.7.7), which function in replication and repair of deoxyribonucleic acid polynucleotides; RNA-dependent DNA polymerases (EC 2.7.7.49), or reverse transcriptases; DNA-dependent RNA polymerases (EC 2.7.7.6), which are primarily responsible for transcription of ribonucleic acid polynucleotides, and also include “primases”, specialized RNA polymerases the synthesize the short RNA primers used in DNA replication; and RNA-dependent RNA polymerases (EC 2.7.7.48), which function primarily in the replication of viral RNA genomes.

Among the most prominent commercial applications of nucleic acid polymerases, are DNA sequencing, e.g., using the Sanger dideoxy termination method (Sanger et al. (1977) Proc Natl Acad Sci USA 74:5463-7), the polymerase chain reaction, in vitro transcription and reverse transcription. Development of specialized enzymes suitable for these applications has relied predominantly on the isolation of novel polymerases with desirable characteristics from natural sources (e.g., isolation of a heat-stable polymerase from Thermus aquaticus) and on mutagenesis at specific sites to improve particular characteristics (e.g., to produce reverse transcriptase lacking RNAse H activity, SuperScript™ (Life Technologies, Rockville, Md.); heat stable Thermo Sequenase® (LiCor, Lincoln, Nebr.). However, the availability of such specialized polymerase is limited and usually involves large investments in either identification and isolation from a natural source (e.g., a thermophilic bacteria) or determination of the three-dimensional structure and characterization of functional groups important for the relevant activity for site specific engineering.

The use of nucleic acid polymerases in a variety of applications is further limited by their generally poor ability to incorporate non-natural and rare nucleotide analogues, including labeled, e.g., fluorescently labeled, nucleotide analogues. The methods of the present invention provide a means for identifying a non-natural or rare nucleotide analogue of interest and generating an enzyme capable of incorporating the nucleotide analogue into a polynucleotide.

Terminal Transferases

A second group of enzymes suitable as substrates in the methods of the present invention are nucleotidyl terminal transferases, or terminal transferases (EC 2.7.7.31). Terminal transferases catalyze the template-independent addition of nucleotides to the 3′ end of a DNA or RNA polynucleotide. In vivo, terminal transferases are involved in adding additional nucleotides during recombination of immunoglobulin and T cell receptor genes. In vitro, terminal transferase have been utilized to add multiple nucleotides, typically homopolymers, to DNA fragments to facilitate, e.g., cloning reactions.

Ligases

Also suitable as substrates for the diversification and selection procedures of the present invention are DNA and RNA ligases. DNA ligases (EC 6.5.1.1, EC 6.5.1.2) covalently join adjacent deoxyribonucleotides in a single strand in the context of a DNA duplex molecule using ATP or NAD+ as a cofactor. In vivo, DNA ligases are involved in joining, e.g., Okazaki fragments generated during replication, discontinuous bases during DNA repair. DNA ligases are also used to mediate assembly of DNA fragments with blunt or overlapping ends, e.g., during cloning of recombinant DNA molecules in vitro.

RNA ligases (EC 6.5.1.3) are primarily involved in processing of tRNA molecules via splicing reactions in vivo and in vitro using ATP as an energy donor.

Telomerases

Nucleic acids encoding telomerases are also suitable substrates for the methods of the invention. Telomerases are ribonucleoproteins with reverse transcriptase activity that synthesize and catalyze the addition of G-rich repeats to the ends of chromosomes. Like the other substrates of the present invention, telomerases catalyze the formation of a phosphodiester bond between a 3′ hydroxyl and a 5′ phosphate moiety.

Nucleic Acid Polymerases

For simplicity, the following discussion focuses primarily on nucleic acid polymerases as substrates for the methods of the present invention. It will be understood that, although, nucleic acid polymerases are one class of preferred embodiments, any of the above described enzyme classes (or their coding nucleic acids) can be used instead of, or in addition to, nucleic acid polymerases in the generation of a diverse library of nucleic acids. Regardless of the substrates elected, the library can be subjected to any of a variety of screening and/or selection procedures aimed at identifying nucleotide incorporating enzymes with an improved capacity to incorporate a non-natural or rare nucleotide analogue into a polynucleotide, i.e., to catalyze the formation of a phosphodiester bond between a non-natural or rare nucleotide analogue and a (poly)nucleotide acceptor, regardless of whether the acceptor is naturally occurring. Unless indicated to the contrary, the term polynucleotide is used to indicate any nucleotide multimer in excess of two nucleotides, including short polynucleotides typically referred to as oligonucleotides, e.g., a nucleotide sequence 5-50 nucleotides in length.

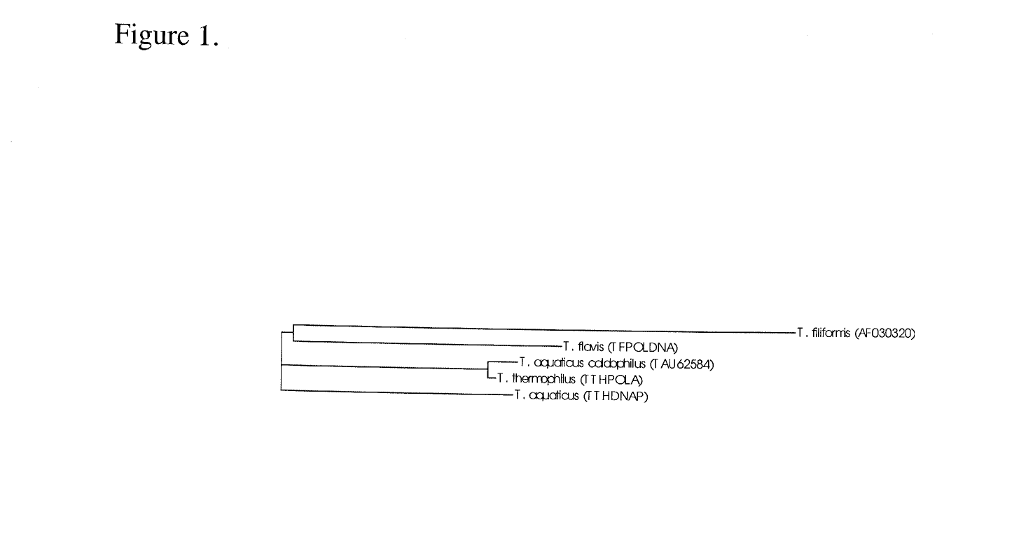

Nucleic acid polymerases catalyze the formation of a phosphodiester bond between a 5′ phosphate moiety of a ribonucleoside or deoxyribonucleoside 5′-triphosphate and a free 3′OH of a sugar of the terminal nucleotide in a polynucleotide. The reaction results in the covalent addition of a nucleotide, typically: adenosine, guanosine, cytidine, thymidine or uridine, to an extending polynucleotide with the release of pyrophosphate (PPi) as illustrated in FIG. 1. Typically, the synthesis of a polynucleotide proceeds from a short polynucleotide (oligonucleotide) primer in the 5′ to 3′ direction in a template dependent manner. Primers and templates that are either DNA or RNA (or DNA and RNA) are employed by various polymerases, and can be appropriately selected by one of skill in the art.

As indicated above, four major classes of nucleic acid polymerases have been described which correspond to synthesis of naturally occurring polynucleotides and are dependent on templates commonly found in nature. As DNA and RNA synthesis are central functions of any living cell or organism, literally thousand of polymerases have been described. Sequences corresponding to the many known polymerases are readily available, e.g., in GenBank™, or other available databases, any or all of which can be used to generate polynucleotide segments, for example, by cloning, PCR or artificial synthesis.

In some cases, it is desirable to utilize nucleic acid segments corresponding to a particular subset, or family, of DNA polymerases in the methods of the invention. Such a choice is influenced by structural and/or functional attributes desired in the recombinant nucleotide incorporating enzyme. Typically, at least a subset of the segments to be recombined or mutated are chosen based on their structural or functional attributes. If it is desirable, segments derived from, or corresponding to, members of multiple families of polymerases, and/or other nucleotide incorporating enzymes can be diversified, e.g., recombined and/or mutated, to generate a library of nucleic acids encoding recombinant nucleotide incorporating enzyme variants.

In vitro applications using DNA-dependent DNA polymerases include, polymerase chain reaction (PCR) using thermostable DNA polymerases, e.g., Taq, Pfu, Tth, Vent®; DNA sequencing typically using the E. coli Polymerase I, Large (Klenow) fragment, or variants thereof; random priming and nick translation to generate labeled probes, e.g., for hybridization; blunting of 5′ and 3′ overhangs left by digestion with a restriction endonuclease to facilitate cloning; and second strand synthesis in site-directed mutagenesis and cDNA production. The known prokaryotic and eukaryotic DNA-dependent DNA polymerases can be divided into several sub-classes based on structural relationships and function, as summarized in Table 1.

TABLE 1
DNA Polymerases

| Source | Polymerase | Role |
|---|---|---|
| Prokaryotic | Polymerase I | excision repair |
| | Polymerase II | DNA repair |
| | Polymerase III | de novo DNA synthesis |
| Eukaryotic | Polymerase α | Nuclear DNA replication |
| | Polymerase δ | |
| | Polymerase ε | |
| | Polymerase β | base excision repair |
| | Polymerase γ | mitochondrial DNA synthesis |

In addition to the DNA-dependent DNA polymerases, RNA-dependent DNA polymerase, or reverse transcriptases have been well described. Reverse transcriptases typically function in nature during the life cycle of retroviruses (and other retrotransposable elements) to synthesize DNA intermediates using an RNA template. Reverse transcriptases typically also possess DNA-dependent DNA polymerase activity as well as RNaseH activity, although engineered variants have been produced which lack RNase H activity, e.g., SuperScript II RNaseH⁻ (Life Technologies, Inc.). Among the applications for which reverse transcriptases are highly desirable are synthesis of cDNA (e.g., first strand synthesis) from RNA templates and copying long mRNA molecules.

DNA-dependent RNA polymerases have been widely used in vitro to generate RNA reagents, such as RNA for in vitro translation and structural studies, labeled RNA probes for hybridization, generation of expression controls using anti-sense RNA molecules. This class of enzymes catalyzes the synthesis of RNA polynucleotides complementary in sequence to a DNA template. In prokaryotes, a single multi-subunit “core” enzyme is functionally modified by interaction with ancillary factors, e.g., sigma (σ); rho (ρ), that help to determine transcriptional specificity and processivity. Eukaryotes have three functionally distinct polymerases, each of which is made up of multiple subunits, and each of which transcribes a different class of genes. For example, RNA polymerase I transcribes all rRNAs except the 5S rRNA. RNA polymerase II transcribes all other RNA molecules, including messenger RNAs, with the exception of certain small RNAs that are transcribed by RNA polymerase III, e.g, tRNAs, 5S rRNA. One specialized subset of DNA-dependent RNA polymerases is the primases, which synthesize short RNA primers used in DNA replication.

RNA-dependent RNA polymerases include viral replicases and/or transcriptases, and are involved in the replication of RNA genomes of RNA viruses and bacteriophages. This class of enzymes is useful, for example, in the in vitro production of infectious RNA transcripts, e.g., for plant transformation procedures.

In addition to the nucleic acid polymerases described above, it is frequently desirable to have nucleotide incorporating enzymes that combine the attributes of one or more nucleic acid polymerase (e.g., fidelity to a template, processivity, activity under specified reaction conditions, among many others) with functional activities of another (one or more) nucleotide incorporating enzyme class.

For example, nucleotidyl terminal transferases, or terminal transferases are widely used to add homopolymer tails to the 3′ end of polynucleotides and for end labeling of probes (especially short, e.g., oligonucleotide probes) for hybridization. Terminal transferases catalyze the addition of nucleotides in a template independent fashion, regardless of whether the polynucleotide is single or double stranded, or whether the end is blunt, recessed or protruding. By using nucleic acid segments derived from both polymerases and terminal transferase, e.g., in a recombination procedure, the methods of the invention are used to produce nucleotide incorporating enzyme variants that exhibit both polymerase and terminal transferase activities.

Nucleotide Incorporating Enzymes That Effeciently Incorporate Non-Natural of Rare Nucleotide Analogues

A significant drawback of currently available nucleic acid polymerases, is that they incorporate non-natural and rare nucleotides; that is, nucleotides other than adenosine, guanosine, cytidine, thymidine or uridine, with poor efficiency. As a result of the low frequency of incorporation, benefits imparted by a non-natural nucleotide, e.g., detectability, resistance to nucleases, stability, etc., cannot readily be conferred upon the synthesized polynucleotide. For example, due to the stochastically low incidence of incorporation of non-natural and rare nucleotide analogues, such nucleotide analogues are incorporated unevenly across the polynucleotide, resulting, e.g., in uneven peak heights in automated sequencing applications.

One particularly desirable application for the nucleotide incorporating enzymes of the invention, is in the generation of polynucleotides that are resistant to degradation under Matrix-assisted laser desorption/ionization (MALDI) mass spectrometry (MS) conditions. Naturally occurring nucleotides are unstable under the high energy, ionizing conditions required for MALDI-MS sequencing methods, making it impossible to sequence polynucleotides in excess of about 100 nucleotides (Wu et al. (1994) Anal Chem 66:1637-1645). By incorporating non-natural base analogues that are less susceptible to degradation under these conditions (e.g., by a modified T7 DNA polymerase lacking 3′-5′ exonuclease activity), longer polynucleotides can be sequenced effectively using this rapid cost-effective procedure. The methods of the invention provide nucleotide incorporating enzymes that can perform this function.

Typically, the nucleotide analogues of interest in the context of the present invention are incorporated at less than about 10%, about 5%, about 1%, about 0.05%, about 0.01%, about 0.05%, about 0.01% (or less), the efficiency at which a naturally occurring nucleotide, e.g., adenosine, guanosine, cytidine, thymidine or uridine, is incorporated by an existing polymerase, such as E. coli DNA polymerase I, Klenow fragment, or other reference polymerase, e.g., a parental polymerase. Examples of nucleotide analogues favorably employed in the context of the present invention include: nucleotides derivatized with a functional group, e.g., a methyl or other alkyl group, a nitrile, formyl, carbonyl, carboxy, halogen, nitroso, or aryl group; nucleotides comprising unnatural base analogues; nucleotides comprising fluorescent labels; nucleotides comprising ribose or deoxyribose analogues; nucleotides comprising an unnatural glycosidic linkage to a base, and nucleotides with unnatural backbone chemistry. The precise composition or identity of the nucleotide analogue is not critical to the invention, as any nucleotide analogue of interest can be evaluated according to, e.g., the methods described herein, to determine whether it is poorly incorporated (i.e., at an efficiency of less than about 10%, or about 5%, about 1%, about 0.05%, about 0.01%, about 0.005%, about 0.001%, or less, the efficiency of a naturally occurring nucleotide).

Similarly, a rare nucleotide (nucleotide analogue), that is, a nucleotide or nucleotide analogue that is incorporated at less than about 10%, or less than about 5%, about 1%, about 0.05%, about 0.01%, about 0.005%, about 0.001%, or less, the frequency of the naturally occurring nucleotides adenosine, guanosine, cytidine, thymidine or uridine, can be identified according to the described methods, e.g., by assaying polynucleotides synthesized by a reference, e.g., a parental, polymerase, for inclusion of the nucleotide or nucleotide analogue of interest.

For example, the nucleotide analogue 7-deaza GTP, and the rare nucleotide inosine, have been utilized in various applications to improve, e.g., evenness of incorporation, resistance to degradation of synthesized polynucleotides, etc. While these nucleotide analogues are incorporated with reduced efficiency with respect to adenosine, guanosine, cytidine, thymidine or uridine, they are, nonetheless, incorporated more efficiently than many of the nucleotide analogues of interest in the present invention.

The present invention offers the significant benefit that the methods can be employed to produce nucleotide incorporating enzymes that incorporate a wide variety of different nucleotide analogues, regardless of the efficiency at which such a nucleotide analogue is incorporated by existing enzymes.

Nucleotide Analogues

Nucleotide analogues useful in the context of the present invention, include a wide range of non-natural and rare nucleotide species. Typically, a nucleotide is selected based on the desirable properties that it confers upon a polynucleotide of which it is a component. For example, various nucleotide analogues confer detectability, e.g., by fluorescent or optical detectors (e.g., microscopes, CCD cameras, plate readers, and the like), stability, e.g., under high energy conditions required for mass spectrometry, resistance to nucleases, among many other useful and desirable properties. Accordingly, nucleotide analogues that are favorably employed in the context of the invention include: nucleotides derivatized with a functional groups, such as methyl or nitrile groups (e.g., 4-methyl-dCTP, 5-methyl-dCTP, 6-methyl-dATP, 7-methyl-dGTP, 7-AZA-dTTP, etc.). Nucleotides comprising unnatural base analogues (e.g.,) are also suitable for use in the context of the present invention, as are nucleotides with unconventional phosphate substitutions, such as phosphorothioate dNTPs, 5′-α-borano-dNTPs, α-methyl-phosphonate dNTPs. Nucleotides comprising isotopic (e.g., ³²P, ³³P, ³⁵S, or the like) or fluorescent labels, such as fluorescein family dyes (e.g., fluorescein, BODIPY, etc.), rhodamine family dyes, cyanine family dyes, as well as nucleotides with other labels, such as, haptens (e.g., biotin), enzymes (e.g., streptavidin, avidin), or pro-fluorescent fluorophores, (especially analogues modified at the 2′ or 3′ hydroxyl position). In addition, nucleotides comprising a ribose or deoxyribose analogue (e.g., 6-(β-Dribofuranosyl)-3,4-dihydro-8H-pyrimido[4,5-c][1,2]oxazin-7-one), and/or nucleotides comprising an unnatural glycosidic linkage to a base are also suitable for the use in the context of the present invention. Nucleotides including bases with backbone modifications such as those introducing novel chiral centers (e.g., phosphothioates, methylphosphonates, etc.) are also suitable for use in the present invention. In this case, the challenge becomes to either perform an enantioselective synthesis, or to use the synthetic diastereomeric mixture such that enzymatic incorporation proceeds stereoselectively, yielding enantiomeric pure oligomers. Another particularly favorable group of nucleotide analogues are those derivatized at the 2′ or 3′ position with any of the above derivatizing and/or labeling agents.

Numerous such nucleotide analogues are known, and available commercially, e.g., from Molecular Probes (Eugene, Oreg.), Glen Research (Sterling, Va.), Fluka BioChemika (Milwaukee, Wis.) or any of a variety of other commercial vendors. Additional favorable nucleotide analogues can be produced by incorporating, e.g., fluorescent moieties, using available technologies.

Diversification of Nucleic Acids Encoding Nucleotide Incorporating Enzymes

Typically, nucleotide incorporating enzyme variants produced by the methods of the invention demonstrate a significant increase in efficiency relative to a reference polymerase, such as the one or more parental polymerases from which the starting materials are derived. For example, in general, a nucleotide incorporating enzyme variant produced by the methods of the invention incorporates a nucleotide analogue of interest that is only poorly incorporated by the reference enzyme (e.g., at less than about 10% or less than about 5%, or less, the efficiency of a naturally occurring nucleotide) with an efficiency of at least 10% the efficiency of a naturally occurring nucleotide. Alternatively, a nucleotide incorporating enzyme variant of the invention incorporates a nucleotide analogue that is incorporated at an efficiency of less than about 10%, less than about 5%, about 1%, about 0.05%, about 0.01%, or less, with an efficiency that is improved at least about 10 fold, about 20 fold, about 50 fold or about 100 fold, or more.

The methods of the invention involve diversification of nucleic acid segments, whether RNA or DNA polynucleotides, or character strings representing RNA or DNA polynucleotides, corresponding to all or part of one or more parental nucleotide incorporating enzyme, e.g., a nucleic acid polymerase, a terminal transferase, a ligase, or a telomerase, followed by selection for efficient incorporation of the nucleotide analogue of interest.

A variety of diversity generating protocols are available and described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, typically a library of nucleic acids, e.g., encoding nucleotide incorporating enzyme variants, which can be selected or screened for nucleic acids that encode proteins with or which confer desirable properties. Following diversification by one or more of the methods herein, or otherwise available to one of skill, any nucleic acids that are produced can be selected for a desired activity or property, e.g. the ability to incorporate non-natural or rare nucleotide analogues into an elongating polynucleotide. This can include identifying any activity that can be detected, for example, in an automated or automatable format, by any of the assays in the art, such as mass spectrometry, fluorescent or optical spectroscopy, in vivo complementation, etc., as described below. A variety of related (or even unrelated) properties can be evaluated, in serial or in parallel, at the discretion of the practitioner.

Descriptions of a variety of diversity generating procedures for generating modified nucleic acid sequences encoding nucleotide incorporating enzymes that incorporate non-natural or rare nucleotides, and/or exhibit other desirable properties are found the following publications and the references cited therein:: Soong, N. et al. (2000) “Molecular breeding of viruses” Nat Genet 25(4):436-439; Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundström et al. (1985) “Oligonucleotide-directed mutagenesis by microscale ‘shot-gun’ gene synthesis” Nucl. Acids Res. 13: 3305-3316), double-strand break repair (Mandecki (1986) “Oligonucleotide-directed double-strand break repair in plasmids of Escherichia coli: a method for site-specific mutagenesis” Proc. Natl. Acad. Sci. USA, 83:7177-7181; and Arnold (1993) “Protein engineering for unusual environments” Current Opinion in Biotechnology 4:450-455). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications and applications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination;” WO 00/18906 by Patten et al., “Shuffling of Codon-Altered Genes;” WO 00/04190 by del Cardayre et al. “Evolution of Whole Cells and Organisms by Recursive Recombination;” WO 00/42561 by Crameri et al., “Oligonucleotide Mediated Nucleic Acid Recombination;” WO 00/42559 by Selifonov and Stemmer “Methods of Populating Data Structures for Use in Evolutionary Simulations;” WO 00/42560 by Selifonov et al., “Methods for Making Character Strings, Polynucleotides & Polypeptides Having Desired Characteristics;” WO 01/23401 by Welch et al., “Use of Codon-Varied Oligonucleotide Synthesis for Synthetic Shuffling;” and PCT/US01/06775 “Single-Stranded Nucleic Acid Template-Mediated Recombination and Nucleic Acid Fragment Isolation” by Affholter.

In brief, several different general classes of sequence modification methods, such as mutation, recombination, etc., are applicable to the generation and selection of enzyme variants that utilize non-natural or rare nucleotides in the synthesis or extension of a polynucleotide, and set forth, e.g., in the references above.

The following exemplify some of the different types of preferred formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Natl. Acad. Sci. USA 91:10747-10751. Thus, nucleic acids encoding all or part of one or more nucleic acid polymerase, terminal transferase, or other nucleotide incorporating enzyme, can be fragmented by enzymatic, chemical or mechanical means, and recombined in vitro to produce a population, e.g., a library, of recombinant nucleic acids encoding nucleotide incorporating enzymes with improved ability to incorporate non-natural and/or rare nucleotide analogues into a polynucleotide.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above. Using any one or more of these procedures, nucleic acids corresponding to one or more nucleotide incorporating enzyme can be transfected into a suitable host cell population and recombined in vivo.

Whole genome recombination methods can also be used in which whole genomes of cells or other organisms are recombined, optionally including spiking of the genomic recombination mixtures with desired library components (e.g., genes corresponding to nucleotide incorporating enzymes, such as nucleic acid polymerases, terminal transferases, ligases, telomerases, and the like). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., PCT/US99/15972 by del Cardayre et al., also entitled “Evolution of Whole Cells and Organisms by Recursive Sequence Recombination.”

Synthetic recombination methods can also be used to diversify nucleic acid segments encoding nucleotide incorporating enzymes. For example, oligonucleotides corresponding to targets one or more nucleotide incorporating enzyme are synthesized and reassembled in PCR or ligation reactions which include oligonucleotides which correspond to more than one parental nucleic acid, e.g., encoding a parental polymerase, terminal transferase, ligase or telomerase, thereby generating new recombined nucleic acids. Alternatively, synthetic oligonucleotides can be joined using only a ligase, in the absence of a polymerase, for example, in a single cycle synthesis reaction. Oligonucleotides can be made by standard nucleotide addition methods, or can be made, e.g., by tri-nucleotide synthetic approaches. Details regarding such approaches are found in the references noted above, including, e.g., “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-BASED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202); “METHODS OF POPULATING DATA STRUCTURES FOR USE IN EVOLUTIONARY SIMULATIONS” by Selifonov and Stemmer (PCT/US00/01138), filed Jan. 18, 2000; and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579).

In synthetic formats, a plurality of oligonucleotides are synthesized which encode a plurality of genes. Typically the oligonucleotides collectively encode sequences derived from homologous parental genes. For example, homologous genes of interest are aligned using a sequence alignment program such as BLAST (Altschul et al., J. Mol. Biol., 215:403-410 (1990)). Nucleotides corresponding to amino acid variations between the homologues are noted. These variations are optionally further restricted to a subset of the total possible variations based on covariation analysis of the parental sequences, functional information for the parental sequences, selection of conservative or non-conservative changes between the parental sequences, or by any other criteria. Variations are optionally further increased to encode additional amino acid diversity at positions identified by covariation analysis of the parental sequences, functional information for the parental sequences, selection of conservative or non-conservative changes between the parental sequences, apparent tolerance of a position for variation or by any other criteria. The result is a degenerate gene sequence encoding a consensus amino acid sequence derived from the parental gene sequences, with degenerate nucleotides at positions encoding amino acid variations. Oligonucleotides are designed which contain the nucleotides required to assemble the diversity present in the degenerate gene sequence.

In one ligase-based format, a plurality of partially duplexed oligonucleotides (i.e., partially hybridized oligonucleotides) having overhangs of unhybridized regions is provided, which oligonucleotides optionally comprise a subsequence of a nucleic acid encoding a nucleotide incorporating enzyme. The partially duplexed oligonucleotides are allowed to hybridize to each other through the unhybridized overhang regions, and are ligated together to produce one or more recombinant nucleic acid(s). The recombinant nucleic acid(s) typically encode a full length protein (although ligation can also be used to make libraries of partial nucleic acid sequences which can then be recombined, e.g., to produce a partial or full-length recombinant nucleic acid). The partially duplexed oligonucleotides may be reassembled with a ligase only, i.e., without a polymerase (e.g., the oligonucleotides may be pre-designed so that no gaps form upon hybridization of the overhangs), or alternatively, a polymerase can optionally be used to extend each strand into any gapped regions to facilitate ligation. Such format is also optionally used as a method for identifying a nucleotide incorporating enzyme having a desired property, the method comprising (a) providing a plurality of partially duplexed oligonucleotides having overhangs of unhybridized regions, which oligonucleotides comprise a subsequence of a nucleic acid encoding a nucleotide incorporating enzyme, (b) assembling the plurality of partially duplexed oligonucleotides by hybridizing the overhangs of two or more partially duplexed oligonucleotides together, (c) ligating the assembled oligonucleotides to produce a library of recombinant nucleic acids, optionally in the presence of a polymerase, (d) expressing the recombinant nucleic acids to generate a library of nucleotide incorporating enzyme variants, and (e) screening the library of nucleotide incorporating enzyme variants for one or more desired property (e.g., incorporation of rare or non-natural nucleotides, thermostability, evenness of nucleotide incorporation, efficient terminal transferase activity, low fidelity, high fidelity, processivity, strand-displacement activity, nick translation activity, exchange reaction, cation requirement, modulation of activity by cation, sulfhydral reagent requirement, shelf life, salt tolerance, organic solvent tolerance, mechanical stress tolerance, tolerance to impurities, altered pH dependence, altered dependence on buffer conditions, template composition, primer composition, and improved stability).

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/gene reassembly techniques. This approach can generate random, partially random or designed variants. The present invention provides a method for producing a recombinant nucleic acid that encodes a nucleotide incorporating enzyme, the method comprising: (a) providing a plurality of parental character strings corresponding to a plurality of nucleic acids, which character strings, when aligned for maximum identity, comprise at least one region of heterology, wherein at least one of the plurality of nucleic acids encodes a parental nucleotide incorporating enzyme or a homologue thereof; (b) aligning the character strings; (c) defining a set of character string subsequences, which set of subsequences comprises subsequences of at least two of the plurality of parental character strings; (d) providing a set of oligonucleotides corresponding to the set of character string subsequences; (e) annealing the set of oligonucleotides; and (f) elongating one or more members of the set of oligonucleotides with a polymerase, or ligating at least two members of the set of oligonucleotides with a ligase, thereby producing one or more recombinant nucleic acid.

Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) “METHODS OF POPULATING DATA STRUCTURES FOR USE IN EVOLUTIONARY SIMULATIONS” by Selifonov and Stemmer (PCT/US00/01138), filed Jan. 18, 2000; and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579). Extensive details regarding in silico recombination methods are found in these applications. This methodology is generally applicable to the present invention in providing for recombination of the nucleotide incorporating enzyme encoding sequences in silico and/or the generation of corresponding nucleic acids or proteins.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “Single-Stranded Nucleic Acid Template-Mediated Recombination and Nucleic Acid Fragment Isolation” by Affholter, PCT/US01/06775.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

In some circumstances, it is desirable, whether recombining nucleic acid segments in vitro, in vivo, or in silico, to employ nucleic acid segments that encode partial or inactive proteins or polypeptides. For example, the recovery of recombinant nucleic acids relative to parental nucleic acids can be significantly increased by providing only parental nucleic acid segments encoding inactive polypeptides or polypeptide fragments. Upon introduction into a host cell, and expression for screening, only recombinants encoding active polypeptide or protein variants will be detected.

In some cases, codon altered nucleic acid segments encoding one or more parental nucleotide incorporating enzyme are favorably employed. Codon altered nucleic acids are frequently preferred, for example, when the ultimate expression host for an enzyme variant differs from the source organism of the parental nucleic acid/enzyme. It is well known in the art, that due to the redundancy of the genetic code, e.g., as illustrated in Table 2, many so-called “silent” mutations, or codon alterations, can be introduced into a coding sequence without altering the encoded polypeptide. Additional details regarding recombination of codon altered nucleic acids can be found in, e.g., PCT/US99/22588, “SHUFFLING OF CODON ALTERED GENES” by Patten et al., filed Sep. 28, 1999.

Another approach involves the introduction of introns or inteins, that is, sequences that are autonomously removed from the encoded polypeptide at the transcriptional or post-translational level, respectively. Introduction of introns and/or inteins, including artificial introns and inteins, is particularly useful, for example, in modulating recombination between sequences, e.g., from different sequence families, such as nucleic acids encoding members of different nucleic acid polymerase classes, with little sequence similarity. Further details regarding intron and intein mediated recombination are found in, e.g., U.S. Ser. No. 60/164,617, “RECOMBINATION OF INSERTION MODIFIED NUCLEIC ACIDS” by Patten et al. filed Nov. 10, 1999.

TABLE 2
Codon Table

| | 2^{nd} position | | | | |
|---|---|---|---|---|---|
| 1^{st} position | U | C | A | G | 3^{rd} position |
| U | Phe | Ser | Tyr | Cys | U |
| | Phe | Ser | Tyr | Cys | C |
| | Leu | Ser | STOP | STOP | A |
| | Leu | Ser | STOP | Trp | G |
| C | Leu | Pro | His | Arg | U |
| | Leu | Pro | His | Arg | C |
| | Leu | Pro | Gln | Arg | A |
| | Leu | Pro | Gln | Arg | G |
| A | Ile | Thr | Asn | Ser | U |
| | Ile | Thr | Asn | Ser | C |
| | Ile | Thr | Lys | Arg | A |
| | Met* | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids. While in some circumstances, it is desirable to begin the diversification with nucleic acids corresponding to a single parental nucleotide incorporating enzyme, it is frequently preferable to use a set or “family” of related, e.g., homologous and/or orthologous, nucleic acids corresponding to multiple members of a protein family. This approach permits the introduction of structural and functional features from a variety of sources, e.g., organisms, functional subgroups, etc., and provides beneficial diversity in the initial population of nucleic acid segments. Such an approach is suitable for both homology and non-homology bases recombination procedures, and is described in detail in the cited references. In addition, it is sometimes desirable to incorporate functional or structural elements from more than one family of sequences, for example, DNA polymerases and terminal transferases. Where sequence similarity is insufficient to mediate recombination, artificial sequences can be introduced, e.g., using intron or intein mediated recombination. Alternatively, non-homology based recombination procedures such as oligonucleotide and/or in silico recombination methods are employed to generate recombinant nucleic acids incorporating elements from multiple sequence families.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See, also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Mutational methods which result in the alteration of individual nucleotides or groups of contiguous or non-contiguous nucleotides can be favorably employed to introduce nucleotide diversity into nucleic acids encoding nucleotide incorporating enzymes. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “METHODS FOR GENERATING AND SCREENING NOVEL METABOLIC PATHWAYS,” and Thompson, et al. (1998) U.S. Pat. No. 5,824,485 METHODS FOR GENERATING AND SCREENING NOVEL METABOLIC PATHWAYS) and their use to identify protein activities of interest has been proposed (In addition to the references noted above, see, Short (1999) U.S. Pat. No. 5,958,672 “PROTEIN ACTIVITY SCREENING OF CLONES HAVING DNA FROM UNCULTIVATED MICROORGANISMS”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215: 471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “PRODUCTION OF ENZYMES HAVING DESIRED ACTIVITIES BY MUTAGENESIS.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods can be applied to the present invention as well.

It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd. (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

Identification of Nucleotide Incorporating Enzymes That Incorporate Non-Natural Nucleotide Analogues

Nucleotide incorporating enzyme variants capable of incorporating non-natural and rare nucleotide analogues can be identified using a variety of in vivo and in vitro techniques.

In many cases, it is desirable to eliminate variants that are unable to synthesize polynucleotides from further consideration using an in vivo selection procedure. For example, by transforming library members encoding nucleotide incorporating enzyme variants into a recA⁻, DNA polymerase I temperature-sensitive E. coli, library members that encode functional DNA-dependent DNA polymerases can be identified (Sweasy and Loeb (1992) J Biol Chem 267(3):1407-10). Due to the high background in this assay due to phenotypic reversion (approximately 1%), it is sometimes desirable to recover the library members from selected colonies and retransform new bacteria which are then subjected to a second round of selection to reduce the false positive rate (i.e., to about 1 in 10,000).

Similarly, library members can be transformed into any host cell population which demonstrates growth dependence on the presence of a nucleotide in the medium, e.g., host cells deficient for a nucleotide synthetic enzyme. By substituting the non-natural or rare nucleotide analogue of interest for the required nucleotide in the growth medium, it is possible to select transformants which express a nucleotide incorporating enzyme that is capable of utilizing the supplied non-natural or rare nucleotide analogue.

Alternatively, or in addition to in vivo selection methods, characterization of polynucleotides produced by a nucleotide incorporating enzyme is also used to identify nucleotide incorporating enzymes that are able to incorporate non-natural or rare nucleotide analogues. For example, any of the following types of assays are favorably employed to detect the nucleotide incorporating enzyme variants of the invention: mass spectrometry, optical or fluorescent spectroscopy, radiometry, chromatography, gel electrophoresis, capillary electrophoresis, streptavidin binding, hybridization, fluorescent resonance energy transfer, fluorescent polarization, and pyrophosphate detection. Any one of the above methods, singly or in combination can be used to identify nucleotide incorporating enzyme variants that incorporate the nucleotide analogue of interest by detecting polynucleotides that include the nucleotide analogue.

For example, mass spectrometry (or spectroscopy) is a generic method that allows detection of a large variety of different small molecule metabolites, including various nucleotides, nucleotide analogues, and polynucleotides. For example, tandem mass spectrometry uses the fragmentation of precursor ions to fragment ions within a triple quadrupole Mass spectrometer (MS). The separation of compounds with different molecular weights occurs in the first quadrupole by the selection of a precursor ion. The identification is performed by the isolation of a fragment ion after collision induced dissociation of the precursor ion in the second quadrupole. Reviews of this technique can be found in Kenneth et al. (1988) Techniques and Applications of Tandem Mass Spectrometry, VCH Publishers, Inc. Additional details regarding the application of mass spectrometry to high-throughput analysis of enzyme variants are provided in, e.g., U.S. patent application “HIGH THROUGHPUT MASS SPECTROMETRY” by Raillard et al. filed Feb. 11, 2000 (Attorney docket Number 2-295-1).

Another approach favorably employed in the context of the present invention, involves characterization of nucleotide incorporating enzyme activity by monitoring pyrophosphate (PPi) activity (Capaldi, S., Getts, R. C., Jayasena, S. D. (2000) Nucleic Acids Res 28:E21). Several coupled enzyme detection systems are well suited for monitoring PPi production upon nucleotide utilization. For example, one method involves colorimetric monitoring of the breakdown of PPi to Pi with pyrophosphatase. Purine ribonucleoside phosphorylase (PNP) subsequently catalyzes substitution of the purine base of, e.g., 2-amino-6-mercapto-7methylpurine ribonucleoside with phosphate. The difference in absorbance spectra of the purine and the corresponding nucleoside is used to monitor phosphate levels. The detection limit of this system is approximately 1 μM PPi. Another approach relies on the utilization of maltose phosphorylase and glucose oxidase to couple H₂O₂ production by horseradish peroxidase to phosphate and Amplex Red to fluorimetrically detect H₂O₂. This system has a detection limit of approximately 0.1 μM PPi. Alternatively, PPi can be coupled to luminescence using ATP sulfurylase to generate ATP from PPi and adenosine 5′-phosphosulfate and firefly luciferase to convert ATP to light. Such a system is highly sensitive, detecting PPi in the sub-nanomolar range. Typically, the above methods involve some form of enzyme purification, such as His-tag affinity binding, prior to the assay to remove phosphate and other small molecules, as well as other enzymes which can interfere or contribute to background.

In the case of nucleotide analogues labeled with fluorophores (or radio-isotopes), incorporation of the fluorophore (or isotope)-labeled nucleotide into a biotin-labeled oligonucleotide by determining the amount of fluorescence (or radioactivity) recovered upon binding of the biotin-conjugated oligonucleotide to avidin (or streptavidin) using an appropriate detector (e.g., a fluorescent plate reader or scintillation detector). This type of assay is particularly favorable for detection of enzyme variants that incorporate fluorescently labeled, e.g., at the 2′ hydroxyl position, nucleotide analogues. These assays are generally not dependent on purification or concentration of the expressed enzyme variant. Rather, such assays involve partitioning of the product following reaction. Typically, partitioning is accomplished in a high-throughput format using avidin-coated microplates or magnetic beads.

An alternative method involves the incorporation of a fluorophore-labeled nucleotide analogue into a large, rigid oligomer, for example, by immobilizing the substrate oligonucleotide on a surface or a bead. Incorporation of the nucleotide analogue is detected as a change in fluorescence polarization (Chen, X., Levine, L., Kwok, P. Y. (1999) Genome Res 9, 492-8). As with detection of incorporation into biotin-labeled oligonucleotides, specificity can be altered by changing the nucleotide analogue. This approach offers the additional advantages that no enzyme purification is required, and post-reaction separation can be eliminated, thus, the assay can be fully automated in a high throughput format.

Similarly, the substrate oligonucleotide can be labeled with a fluorescent resonance energy transfer (FRET) partner of the nucleotide analogue (Norman, D. G., Grainger, R. J., Uhrin, D., Lilley, D. M. (2000) Biochemistry 39, 6317-24. Vitiello, D., Pecchia, D. B., Burke, J. M. (2000) RNA 6, 628-37). Emission of the acceptor upon excitation of the donor, or quenching of the donor, can be used to monitor nucleotide incorporation. This assay can be completely automated, and is likely to offer the highest-throughput of all the formats discussed.

Fluorescence-assisted cell sorting can be used to screen for polymerases capable of incorporating rare or novel nucleotides by co-displaying the polymerase with a double-strand DNA binding protein on a cell, phage, or viral surface. Single-strand DNA template that is successfully extended by the displayed polymerase in the presence of a fluorescent nucleotide would be bound by the co-displayed DNA binding protein. Streptavidin may be co-displayed with the polymerase and a biotin-labeled template used as an alternative to the DNA binding protein.

After initial screening in one of the high-throughput formats, the variants identified can be more thoroughly characterized using variations of the assays described above to determine, e.g., substrate specificity, condition dependence, and specific activity.

Identification of Nucleotide Incorporating Enzymes with Desired Properties

The methods diversity generating procedures described above can also be used to identify nucleotide incorporating enzymes with one or more desired property, optionally coupled with the ability to efficiently incorporate a non-natural or rare nucleotide analogue of interest. For example, any one or more of the following properties can be screened or selected for using a variety of methods known to one of skill in the art, and chosen according to the specific property desired, e.g., thermostability, evenness of nucleotide incorporation, efficient terminal transferase activity, low fidelity, high fidelity, processivity, strand-displacement activity, nick translation activity, exchange reaction, cation requirement, modulation of activity by cation, sulfhydral reagent requirement, shelf life, salt tolerance, organic solvent tolerance, mechanical stress tolerance, tolerance to impurities, altered pH dependence, altered dependence on buffer conditions, template composition, primer composition, and improved stability. An improvement in a desired property may be identified, for example, by comparison to the same property exhibited by one or more of the parent nucleotide incorporating enzymes using screening methods described herein as well as methods that are known to those having skill in the art.

Polymerases That Perform Under Altered Reaction Conditions

T7 RNA polymerase (T7 RNA pol) is the most widely used RNA polymerase for in vitro transcription reactions for both research and diagnostic purposes. It binds to a specific double-stranded 17-mer promoter sequence and synthesizes RNA oligomers using a single- or double-stranded DNA template downstream of the promoter. Typically, each template molecule will be transcribed 50 to 100 times.

T7 RNA pol is rapidly denatured under high temperatures and/or oxidizing environments. For many laboratory and diagnostic applications, a more heat-stable RNA polymerase would be useful. The present invention provides a method for screening for and identifying a thermostable RNA polymerase with increased efficiency (processivity and/or rate) due to its stability at elevated temperatures. Nucleotide incorporating enzyme variant libraries of the present invention may also be screened for polymerase variants that do not undergo termination slippage and that are not dependent on the GG starting sequence for high yield. The methods of the present invention can be used to produce RNA polymerases with these (and other) desirable properties.

The optimal reaction temperature for elongation by Taq DNA polymerase is 72° C. At 25° C. the activity of Taq polymerase is reduced to 20-30% of its normal activity at 72° C. (Perkin Elmer, technical service). Thus, efficient PCR cannot be performed when primer-template binding interaction consists of only a few base pairs, necessitating annealing and extension reactions to occur at lower temperatures. The present invention includes screening for a thermostable polymerase that retains an efficient nucleotide incorporating activity across a wide range of temperatures.

Efficient amplification over long distances (with respect to polynucleotide length) is important for a variety of purposes, including, e.g., diversification of DNA by some shuffling procedures. The effective limit for Taq polymerase is about 4 kb. Kits containing thermostable polymerases with additives (Promega, Stratagene) are able to extend up to 40 and 35 kb, respectively. The nature and mechanism of these additives is unclear. Most likely, these additives are additional enzymes that enable Taq or Tth polymerases to perform long range PCR. The present invention provides for the screening of nucleotide incorporating enzyme variant libraries and isolation of a thermostable nucleotide incorporating enzyme (e.g., a polymerase) that can extend very long DNA molecules without the need of proprietary additives. The present invention further provides for the isolation of thermostable DNA polymerases that yield balanced PCR products from the nucleotide incorporating enzyme variant libraries (i.e., balanced PCR products produced from the enzyme variant libraries will show final product percentages roughly equal to the corresponding variant percentages in the starting library mixture). The enzyme variant libraries of the present invention can also be screened to identify and obtain polymerases that perform PCR in volatile buffers that are amenable to downstream DNA detection by MALDI-TOF or ESI mass spectrometry both for MS sequencing or for MS-dependent SNP analysis.

Assays for evaluating the additional desired property or properties can be performed sequentially or simultaneously, after a single round of diversification or after one or more additional rounds of diversification, as desired by the practitioner. For example, a nucleotide incorporating enzyme variant which exhibits high efficiency incorporation of a specified fluorescently labeled nucleotide analogue, can be screened for the ability to add homopolymer tails, e.g., of the same fluorescently labeled nucleotide analogue, or another nucleotide analogue. Alternatively, variants with increased or decreased template fidelity can be screened or selected, optionally following additional diversification procedures.

To increase throughput in any of the selection assays, it is often desirable to prescreen diversified libraries for active polymerases. The following in vivo selection procedure can be used to eliminate inactive polymerase variants from further consideration in a rapid and cost-efficient manner.

As described by Loeb and coworkers (Sweasy and Loeb, 1992). A recA⁻, DNA polymerase I temperature-sensitive E. coli grows normally at the restrictive temperature only when complemented by another polymerase. So far, mammalian pol β, HIV reverse transcriptase, and Taq DNA polymerase have been shown to complement this strain, and it is likely that any DNA-dependent DNA polymerase will work. The background in this selection, i.e., normal growth of bacteria in the absence of complementing polymerase, is 1% due to phenotypic reversion, so in some cases it may be desirable to isolate plasmid from the selected colonies, retransform new bacteria, and reselect growing colonies to reduce the false positive rate to 0.01% (1 in 10,000).

Variants that are isolated from the in vivo selection method described above can be screened for any desired phenotype using a high-throughput format. To facilitate purification and subsequent assay, the polymerase variants can be expressed with an 6x-histidine-tag at the N-terminus using an appropriate vector. After induction of protein synthesis, the cells can be harvested by centrifugation and lysed, e.g., by addition of lysozyme, or by using a mechanical method such as sonication or freeze and thaw. The protein can be purified from the crude cellular extract using a Nickel-affinity resin (e.g., Talon resin from Clontech). High-throughput can be achieved, e.g., using membrane based microtiter plates from Millipore. The membranes allow passage of liquid while any solid material is retained in the wells. Liquid is passaged through by centrifugation or by attaching the plate to a vacuum manifold that sucks any liquid through the membrane. The protein is purified as follows using each well of the microtiter plate as a micro-column, in the following manner. Nickel-affinity resin is aliquoted into the wells of the microtiter plate. The wells are then loaded with a crude extract, e.g., derived from a colony of lysed cells. Upon passage of the liquid through the membrane into the well, protein is bound to the resin. The well is then washed and the purified protein is eluted, e.g., with imidazole buffer, into a new microtiter plate. Elution is preferentially performed by centrifugation in order to ensure complete and clean recovery of each sample. The use of conventional robotics allows for purifications in the order of 10⁴ samples within a reasonable time (1-2 days).

High Throughput Assays/Formats

It is often desirable to perform the screening or selection in a high throughput format. Any of the methods described above can be adapted to high throughput formats, as will be apparent to one of skill in the art. High throughput is generally considered to be in excess of 100, frequently in excess of 1000, and often in excess of 10,000 samples per day. Numerous formats for accomplishing high throughput screening are known in the art. Among the more common formats are microtiter plates, pin arrays, bead arrays, membranes, filters and microfluidic devices.

One standard format for the performance of high throughput assays is microtiter plates. Microtiter plates with 96, 384 or 1536 wells are widely available, however other numbers of wells, e.g., 3456 and 9600 are also used. In general, the choice of microtiter plates is determined by the handling and/or analytical device to be used, e.g., automated loading and robotic handling systems. Exemplary systems include the ORCA™ system from Beckman-Coulter, Inc. (Fullerton, Calif.) and the Zymate systems from Zymark Corporation (Hopkinton, Mass.).

Alternatively, other formats such as “chip” or pin arrays, or formats involving immobilization of one or more assay component on a solid support such as a membrane or filter, e.g., nylon, nitrocellulose, and the like, are employed in high throughput assays useful in the context of the present invention. In addition, numerous assays useful in detecting proteins, or cells expressing proteins, with desirable properties can be performed in microfluidic devices such as the Lab Microfluidic Device™ high throughput screening system (HTS) by Caliper Technologies, Mountain View, Calif. or the HP/Agilent technologies Bioanalyzer using LabChip™ technology by Caliper Technologies Corp. See, also, www.calipertech.com.

For example, high-throughput enzyme activity assay can be performed using a similar format as described above for protein purification. Polymerases that are identified as active in the prescreen described above, (or that are otherwise shown to perform primer extension) can be characterized using microtiter plates coated with strepavidine to bind a biotinylated primer. A polynucleotide polymerization reaction occurs with the addition of suitable buffer, purified enzyme variant and, e.g., each of the four dNTP's (nucleotides can be varied depending on the property under consideration, e.g., to include one or more non-natural or artificial nucleotide analogue). Typically, one of the dNTP's is spiked with a trace amount of radioactive [α-³²P]-dNTP. The newly synthesized strand will then be radioactively labeled. After a certain incubation time, the reaction mixture can be washed with high salt and/or detergent to remove any excess [α-³²P]-dNTP that has not been incorporated. Assessment of incorporation of radioactive nucleotides, e.g., by scintillation counting or phosphorimager scanning, is used to determine which of the polymerase variants are able to effectively polymerize a polynucleotide. If desired, incubation temperatures can be varied to adjust to the desired temperature at which polymerization should occur. RNA polymerases can be screened using microtiter plates with hydrophobic resins to which oligomeric nucleotides will bind but not nucleotide triphosphates (e.g., Nensorb reverse phase, DuPont).

To identify polymerases that perform a polymerase chain reaction, e.g., under altered conditions, a thermocycling apparatus that permits amplification reactions in 96 well microtiter plates is favorably employed.

Radioactive labeling assays do not distinguish between variants that perform very long oligonucleotide synthesis (desired phenotype) and those that perform many short syntheses. To distinguish between these two alternatives, variants selected, e.g., according to the method described above, can be loaded onto polyacrylamide or agarose gels, and assayed by electrophoresis. In this manner, it is readily feasible that a single practitioner evaluate greater than about 1000 library members in a single day. For example, a typical gel can be loaded with 96 samples and will need about 40 min. experimental time as follows: (a) loading of 12 samples simultaneously with multichannel pipettor: approximately 10 minutes; (b) electrophoresis: approximately 20 minutes; (c) phosphorimager scanning: approximately 10 minutes, with a single person running from about 10 to 15 gels per day (960 to 1440 samples).

High Throughput Solid Phase Screen for Polymerase Activity

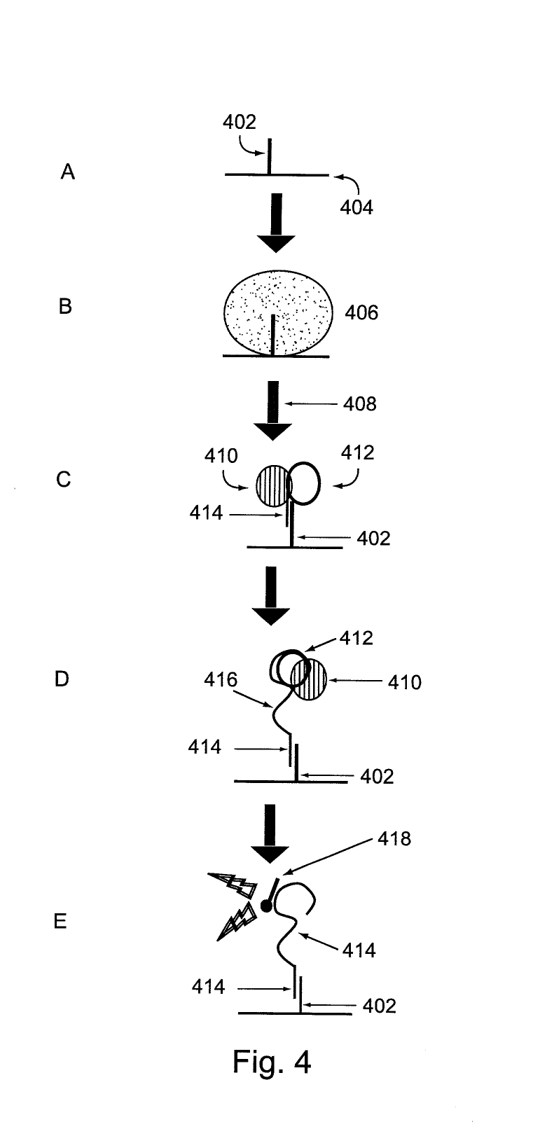

A non-limiting example of high throughput screening as used with the current invention is illustrated in FIG. 4. As shown in FIG. 4, bacterial colonies (406) are grown on membranes (404) that have been modified by immobilizing a specific DNA (402) on the surface of the membrane. Upon polymerase expression and cell lysis (408), a target DNA (412) and a primer (414) are added to the released DNA polymerase (410). The primer hybridizes to the immobilized DNA (see, FIG. 4C) as well as serves as a primer for DNA polymerization using the target DNA as a template. Active DNA polymerases can thus be detected by detecting the extended DNA (416). This is achieved by washing away the target DNA by adding a specific oligonucleotide (418) that is labeled with a fluorescent moiety. After another washing, the fluorescent signal is proportional to the amount of DNA amplified, thus directly proportional to DNA polymerase activity.

Preparation of Nucleic Acids Encoding the Enzymes of the Invention

In general, nucleic acids encoding the enzymes, e.g., nucleotide incorporating enzymes or enzyme variants of the invention can be prepared using various methods or combinations thereof, including certain DNA synthetic techniques (e.g., mononucleotide- and/or trinucleotide-based synthesis, reverse-transcription, etc.), DNA amplification, nuclease digestion, etc. The identification and acquisition of desirable substrate nucleic acids can be facilitated by a variety of means. For example, selection algorithms can be used to identify sequences corresponding to nucleotide incorporating enzymes in public or proprietary databases which meet any user-selected criterion for substrate selection. These user criteria include, activity, encoded activity, homology, public availability, and any other criteria of interest. In addition, character strings corresponding to nucleic acids can be generated according to any set of criteria selected by the user, including similarity to existing sequences, modification of an existing sequence according to any desired modification parameter (genetic algorithm, etc.), random, or non-random (e.g., weighted) sequence generation, etc. Data structures comprising diverse sequences can be formed in a digital or analog computer or in a computer readable medium and the data structures converted from character strings to nucleic acids for subsequent physical manipulations. Either computer data or nucleic acids can be “data structures,” a term which refers to the organization and optionally associated device for the storage of information, typically comprising multiple “pieces” of information. The data structure can be a simple recordation of the information (e.g., a list) or the data structure can contain additional information (e.g., annotations) regarding the information contained therein, can establish relationships between the various “members” (information “pieces”) of the data structure, and can provide pointers or linked to resources external to the data structure. The data structure can be intangible but is rendered tangible when stored/represented in tangible medium. The data structure can represent various information architectures including, but not limited to simple lists, linked lists, indexed lists, data tables, indexes, hash indices, flat file databases, relational databases, local databases, distributed databases, thin client databases, and the like. Nucleic acids can be selected by the user based upon sequence similarity to one or more additional nucleic acids. Different types of similarity and considerations of various stringency and character string length an be detected and recognized during target selection and acquisition. For example, many homology determination methods have been designed for comparative analysis of sequences of biopolymers, for spell-checking in word processing, and for data retrieval from various databases. With an understanding of double-helix pair-wise complement interactions among the principal nucleotides in natural polynucleotides, models that simulate annealing of complementary homologous polynucleotide strings can also be used as a foundation of sequence alignment or other operations typically performed on the character strings corresponding to the sequences of interest (e.g., work-processing manipulations, construction of figures comprising sequence or subsequence character strings, output tables, etc.). An example of a dedicated software package with genetic algorithms for calculating sequence similarity and other operations of interest is BLAST, the Basic Local Alignment Search Tool (BLAST) algorithm, (described in Altschul et al. (1990) J Mol Biol 215:403) which can be used in the present invention to select target sequences (e.g., based upon homology) for acquisition and recombination. Software for performing BLAST analyses is publicly available through the National Center for Biotechnology Information (http://www.ncbi.nlm.nih.gov/).

Searchable sequence information is available from nucleic acid databases can be utilized during the nucleic acid sequence selection and/or design processes. GenBank®, Entrez®, EMBL, DDBJ, GSDB, NDB and the NCBI are examples of public database/search services that can be accessed. These databases are generally available via the internet or on a contract basis from a variety of companies specializing in genomic information generation and/or storage. These and other helpful resources are readily available and known to those of skill. Any one or more of the above referenced resources can be used to identify and/or manipulate nucleotide incorporating enzymes and enzyme variants of the invention.

The sequence of a polynucleotide to be used in any of the methods of the present invention can also be readily determined using techniques well-known to those of skill, including Maxam-Gilbert, Sanger Dideoxy, and Sequencing by Hybridization methods. For general descriptions of these processes consult, e.g., Stryer (1995) Biochemistry (4^{th} Ed.) W. H. Freeman and Company, New York, (“Stryer”) and Lewin (1997) Genes VI Oxford University Press, Oxford (“Lewin”). See also, Maxam and Gilbert (1977) Proc Natl Acad Sci USA 74:560, Sanger et al. (1977) Proc Natl Acad Sci USA 74:5463, Hunkapiller et al. (1991) Science 254:59, and Pease et al. (1994) Proc Natl Acad Sci USA 91:5022.

After sequence information has been obtained as described above, that information can be used to design and synthesize target nucleic acid sequences corresponding to, e.g., overlapping nucleic acid segments encoding nucleotide incorporating enzymes and enzyme variants, or other nucleic acid segments (e.g., for the oligonucleotide and in silico shuffling approaches noted above). These sequences can be synthesized utilizing various solid-phase strategies involving mononucleotide- and/or trinucleotide-based phosphoramidite coupling chemistry. In these approaches, nucleic acid sequences are synthesized by the sequential addition of activated monomers and/or trimers to an elongating polynucleotide chain. See e.g., Caruthers, M. H. et al. (1992) Meth Enzymol 211:3. Additional details are supplied in U.S. patent application Ser. No. 09/408,393 file Sep. 28, 1999 USE OF CODON-BASED OLIGONUCLEOTIDE SYNTHESIS FOR SYNTHETIC SHUFFLING, herein incorporated by reference.

In lieu of synthesizing the desired sequences, essentially any nucleic acid can optionally be custom ordered from any of a variety of commercial sources, such as The Midland Certified Reagent Company (mcrc@oligos.com), The Great American Gene Company (www.genco.com), ExpressGen, Inc. (www.expressgen.com), Operon Technologies, Inc. (www.operon.com), and many others.

Nucleic acids encoding any of the enzymes described above can be derived from expression products, e.g., mRNAs expressed from genes within a cell of a plant or other organism. A number of techniques are available for detecting RNAs. For example, northern blot hybridization is widely used for RNA detection, and is generally taught in a variety of standard texts on molecular biology, including Ausubel. Sambrook et al. Molecular Cloning—A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”). Furthermore, one of skill will appreciate that essentially any RNA can be converted into a double stranded DNA using a reverse transcriptase enzyme and a polymerase. See, Ausubel, Sambrook and Berger. Messenger RNAs can be detected by converting, e.g., mRNAs into cDNAs, which are subsequently detected in, e.g., a standard “Southern blot” format.

Examples of techniques sufficient to direct persons of skill through in vitro amplification methods, useful e.g., for amplifying nucleic acids encoding any of the enzymes of the invention, include the polymerase chain reaction (PCR), the ligase chain reaction (LCR), Qβ-replicase amplification, and other RNA polymerase mediated techniques (e.g., NASBA). These techniques are found in Ausubel, Sambrook, and Berger, as well as in Mullis et al. (1987) U.S. Pat. No. 4,683,202; PCR Protocols A Guide to Methods and Applications (Innis et al. eds.) Academic Press Inc. San Diego, Calif. (1990) (“Innis”); Arnheim and Levinson (1990) C&EN 36; The Journal Of NIH Research (1991) 3:81; Kwoh et al. (1989) Proc Natl Acad Sci USA 86, 1173; Guatelli et al. (1990) Proc Natl Acad Sci USA 87:1874; Lomeli et al. (1989) J Clin Chem 35:1826; Landegren et al. (1988) Science 241:1077; Van Brunt (1990) Biotechnology 8:291; Wu and Wallace (1989) Gene 4: 560; Barringer et al. (1990) Gene 89:117, and Sooknanan and Malek (1995) Biotechnology 13:563. Improved methods of cloning in vitro amplified nucleic acids are described in Wallace et al. U.S. Pat. No. 5,426,039. Improved methods of amplifying large nucleic acids by PCR are summarized in Cheng et al. (1994) Nature 369:684 and the references therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, Ausubel, Sambrook and Berger, all supra.

In one preferred method, assembled sequences are checked, e.g., for incorporation of enzyme encoding nucleic acid subsequences. This can be done by cloning and sequencing the nucleic acids, and/or by restriction digestion, e.g., as essentially taught in Ausubel, Sambrook, and Berger, supra. In addition, sequences can be PCR amplified and sequenced directly. Thus, in addition to, e.g., Ausubel, Sambrook, Berger, and Innis, additional PCR sequencing methodologies are also particularly useful. For example, direct sequencing of PCR generated amplicons by selectively incorporating boronated nuclease resistant nucleotides into the amplicons during PCR and digestion of the amplicons with a nuclease to produce sized template fragments has been performed (Porter et al. (1997) Nucleic Acids Res 25:1611).

Introduction of Nucleotide Incorporating Enzyme Variants into the Cells of Organisms of Interest

In certain embodiments of the present invention, nucleic acid sequences encoding one or more naturally occurring enzyme or enzyme variant are introduced into the cells of particular organisms of interest. There are several well-known methods of introducing target nucleic acids into, e.g., bacterial cells, any of which may be used in the present invention. These include: fusion of the recipient cells with bacterial protoplasts containing the DNA, electroporation, projectile bombardment, and infection with viral vectors, etc. Bacterial cells can be used to amplify the number of plasmids containing DNA constructs of this invention.

Bacteria are typically grown to log phase and the plasmids within the bacteria can be isolated by a variety of methods known in the art (see, for instance, Sambrook). In addition, a plethora of kits are commercially available for the purification of plasmids from bacteria. For their proper use, follow the manufacturer's instructions (see, for example, EasyPrep™, FlexiPrep™, both from Pharmacia Biotech; StrataClean™, from Stratagene; and, QIAexpress Expression System™ from Qiagen). The isolated and purified plasmids are then further manipulated to produce other plasmids.

Numerous well-established methods are known to those of skill in the art for the transfection of yeast, fungal and plant cells. For example, yeast cells can be transfected by preparation of spheroblasts or by treatment with alkaline salts. DNA can be introduced into plant and fungal cells by, for example, electroporation, microinjection, PEG precipitation, or particle-mediated bombardment (“biolistics”). Agrobacterium mediated transformation can be used to introduce exogenous DNA sequences situated between T-DNA ends into plant protoplasts, plant tissue explants, and whole plants as well as fungal cells using appropriate strains and vectors. For a more complete discussion, see, e.g., Jones (ed.) (1995) Plant Gene Transfer and Expression Protocols—Methods in Molecular Biology, Volume 49 Humana Press Towata N.J.; Gamborg and Phillips (eds.) (1995) Plant Cell, Tissue and Organ Culture; Fundamental Methods Springer Lab Manual, Springer-Verlag (Berlin Heidelberg New York) and R. R. D. Croy, Ed. (1993) Plant Molecular Biology Bios Scientific Publishers, Oxford, U.K. In some cases, it is desirable to regenerate a transgenic plant from the transfected protoplast or explant, and techniques for so doing are well-known in the art, see, e.g., Evans et al. (1983) Protoplast Isolation and Culture, Handbook of Plant Cell Culture, Macmillan Publishing Company, New York.

Typically, vectors useful for the transformation of cells and/or organisms contain additional nucleotide sequences, such as transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., replicable vectors) and selection markers for both prokaryotic and eukaryotic systems. Vectors include any vehicle for introducing a subject polynucleotide into a host cell. As such, vectors include non-replicable vehicles such as naked DNA, conjugated DNA, liposomes, and the like. Also included are replicable vectors, such as plasmids, viruses, bacteriophages, cosmids, artificial chromosomes, and the like, for replication and integration in prokaryotes, eukaryotes, or preferably both. See, Giliman and Smith (1979) Gene 8:81; Roberts et al. (1987) Nature 328:731; Schneider et al. (1995) Protein Expr Purif 6435: 10; Ausubel, Sambrook, Berger (all supra). A catalogue of Bacteria and Bacteriophages useful for cloning is provided, e.g., by the ATCC, e.g., The ATCC Catalogue of Bacteria and Bacteriophage Gherna et al. (eds.) (1992) published by the ATCC. Furthermore, a wide variety of cloning kits and associated products are commercially available from, e.g., Pharmacia Biotech, Stratagene, Sigma-Aldrich Co., Novagen, Inc., Fermentas, and 5 Prime→3 Prime, Inc.

Kits

The present invention also provides a kit or system for performing one or more of the polynucleotide synthesis reactions described herein. The kit or system can optionally include a set of instructions for practicing one or more of the methods described herein; one or more assay components that can include at least one recombinant, isolated and/or diversified nucleotide incorporating enzyme variant or at least one cell that includes one or more such enzymes or both, and one or more reagents; and a container for packaging the set of instructions and the assay components. The assay component can optionally include at least one immobilized enzyme as described above, or at least one such enzyme free in solution, or both.

Recombinant, isolated, or diversified nucleotide incorporating enzyme variants, or a combination thereof, can be supplied as assay components (e.g., for a variety of primer extension reactions) of the kits or systems of the present invention, to catalyze the extension of a polynucleotide primer.

In a further aspect, the present invention provides for the use of any component or kit herein, for the practice of any method or assay herein, and/or for the use of any apparatus or kit to practice any assay or method herein.

Integrated Systems

The present invention provides integrated systems for the detection of a polynucleotide product incorporating a non-natural or rare nucleotide analogue. Such a system comprises a non-natural or rare nucleotide analogue, or polynucleotide comprising such a nucleotide analogue, a nucleotide incorporating enzyme variant, e.g., a kit containing a nucleotide incorporating enzyme variant as described above, and a detector. Suitable detectors include: mass spectrometers, optical and/or fluorescent detectors, and the like. The integrated system optionally also comprises one or more of a user input device, a data processing device, a data output device, and a robotic controller for manipulating the enzyme, reactants, e.g., non-natural nucleotide analogue, primer, template, etc., detecting the polynucleotide, and collecting and/or manipulating the results of a detection assay.

The invention also optionally provides computers, computer readable media and integrated systems comprising character strings corresponding nucleic acids encoding nucleotide incorporating enzymes and enzyme variants. These sequences can be manipulated by in silico shuffling methods, or by standard sequence alignment (also discussed, supra), or word processing software.

Thus, integrated systems for analysis in the present invention can include a digital computer with software for aligning or manipulating nucleic acid sequences as well as data sets entered into the software system comprising any of the sequences herein. The computer can be, e.g., a PC (Intel x86 or Pentium chip-compatible DOS™, OS2™ WINDOWS™ WINDOWS NT™, WINDOWS95™, WINDOWS98™ LINUX based machine, a MACINTOSH™, Power PC, or a UNIX based (e.g., SUN™ work station) machine) or other commercially common computer which is known to one of skill. Software for aligning or otherwise manipulating sequences is available, or can easily be constructed by one of skill using a standard programming language such as Visual basic, Fortran, Basic, Java, or the like.

A digital system can also instruct an oligonucleotide synthesizer to synthesize oligonucleotides corresponding to one or more of the naturally occurring or altered nucleotide incorporating enzymes or a substrate thereof, e.g., used for gene reconstruction or recombination, or to order such oligonucleotides from commercial sources (e.g., by printing appropriate order forms or by linking to an order form on the internet).

The digital system can also include output elements for controlling nucleic acid synthesis, i.e., an integrated system of the invention optionally includes an oligonucleotide synthesizer or an oligonucleotide synthesis controller for synthesizing nucleic acid fragments. The system can include other operations which occur downstream from an alignment or other operation performed using a character string corresponding to a sequence herein, e.g., as noted above with reference to assays.

### EXAMPLE

Production of a Thermostable Polymerase with High Tolerance for Biological Impurities

Methods for characterizing RNA expression from biological samples, including medically relevant fluids such as blood, plasma and urine, typically require significant time and effort dedicated to sample preparation and purification. This expenditure is necessitated by the relatively poor ability of existing DNA polymerases to reverse transcribe an RNA template from biological samples in the presence of contaminants. A RNA dependent DNA polymerase capable of reverse transcribing templates from a crude biological sample, such as blood, plasma or urine, in a high throughput format compatible with routine amplification reactions (e.g., PCR) would therefore be of significant utility. In addition, the ability to incorporate dUTP with high efficiency would be a desirable characteristic enabling a variety of automatable detection, monitoring and recovery procedures among other useful applications. Accordingly, one exemplary application of the methods of the invention is the development of a thermostable RNA dependent DNA polymerase capable of reverse transcription in amplification reactions in reaction mixtures containing significant amounts of biological impurities.

Identification of Starting Genes

The DNA polymerase of Thermus thermophilus exhibits reverse transcriptase as well as dUTP incorporation activities (see, U.S. Pat. No. 5,693,517 to Gelfand et al. (Dec. 2, 1997), “Reagents and methods for coupled high temperature reverse transcriptase and polymerase chain reaction”), and is reported to be tolerant to up to 20% (v/v) blood in polymerase chain reactions (PCRs) (Abu Al-Soud & Radstrom (1998) Appl Environ Microbiol. 64, 3748). Accordingly, this and other thermostable DNA polymerases are favorable substrates for the diversification procedures described herein. In one approach, nucleic acid sequences corresponding to published thermostable DNA polymerases (and optionally additional polymerases) provide the starting point for the production of libraries using computational tools (e.g., as described above). Such approaches make it possible to obtain variants that are further away in sequence space from each other as well as from the starting (parental) sequences than is usually encountered among bacterial species.

Alternatively, polymerase (and optionally, other nucleotide incorporating enzyme) genes can be amplified and recovered from genomic DNA of thermophilic organisms of various strains and species. Either of these approaches, independently or in combination, can be used to produce diverse libraries of polymerases from which the desired activities can be recovered.

Production of Libraries

A number of strategies for producing diverse libraries of nucleic acids encoding polypeptides with DNA polymerase activity are available. Any one or more of these approaches can be used to produce libraries for subsequent evaluation as described above. In general, the methods are selected to produce libraries that vary in recombination and/or mutation frequency, and that differ in the introduction of sequence diversity outside the sequence space of the parental or starting sequences.

For example, one in one approach a collection of DNA polymerases from various Thermus species serve as the parental sequences. Both T. aquaticus (Taq) and T. thermophilus (Tth) polymerases show reverse transcriptase activity as well as the ability to incorporate dUTP ((see, U.S. Pat. No. 5,693,517 to Gelfand et al. (Dec. 2, 1997), “Reagents and methods for coupled high temperature reverse transcriptase and polymerase chain reaction”); Jones et al. (1989) Nucleic Acids Res. 17, 8387; and Slupphaug et al. (1993) Anal. Biochem. 211, 164). Typically, recombinatorial procedures, e.g., as described above, using related sequences, multiple members of which possess a desired activity results in a high quality library from which multiple progeny with the desired properties can be obtained. FIG. 1 illustrates the phylogenetic relationship between the amino acid sequences of Pol I DNA polymerases of 5 Thermus species. Pairwise amino acid identities range from 98.9% (T. thermophilus vs. T. aquaticus caldophilus) to 77.4% (T. filiformis vs. T. flavis) indicating that the sequences fall in an ideal range for many of the, e.g., DNA shuffling methods described above.

Alternatively, libraries can be produced from parental sequences that are relatively distant from the Thermus polymerases. For example, several Bacillus species (see, WO 00/71739, Schanke et al., “Reverse transcription activity from Bacillus stearothermophilus DNA polymerase in the presence of magnesium”), as well as other thermophilic organisms (see, WO 98/14588, Ankenbauer, B. et al., “Thermostable DNA polymerase from Anaerocellum thermophilum”; EP 921196, 1999, Ankenbauer et al., “Modified DNA-polymerase from Carboxydothermus hydrogenoformans and its use for coupled reverse transcriptase and polymerase chain reaction”; and U.S. Pat. No. 5,939,301 to Hughes et al. (Aug. 17, 1999 ) “Cloned DNA polymerases from Thermotoga neapolitana and mutants thereof”), have thermostable DNA polymerases with RT activity. Additionally, parents can include DNA polymerases from Pyrococcus and Thermococcus species.

Alternatively, sequence or structural analysis, e.g., computational methods, hybridization, etc., can be used to identify thermostable DNA polymerases that have no reported RT or dUTP incorporation activities but are suitable starting materials based sequence, structural or functional proximity to thermostable polymerases with established RT or dUTP incorporation functions. Similarly, it is also possible to start with one or more DNA polymerase that are not thermostable but exhibit reverse transcriptase activity, e.g., E. coli DNA polymerase I (Richetti & Buc (1993) EMBO J. 12, 387) and evolve for thermostability.

Each of these approaches can be used independently or in combination with these or other methods to produce a diverse nucleic acid sequence library. The quality, i.e., the diversity, frequency of clones encoding polymerase activity, etc., of each library can be assessed prior to extensive screening of any one or more libraries produced as described above.

Expression and Assay Development

Thermostable DNA polymerases are routinely expressed in E. coli, and a number of E. coli expression systems are available that are suitable for this purpose. Any available expression system can readily be evaluated by one of skill in the art, and an expression system which provides a sufficient level of soluble and functional polymerase can be selected empirically.

Following optimization of an expression system, the libraries are evaluated based on functional criteria. Typically, it is advantageous to start with a very high throughput prescreen, and proceed through tiered assays with progressively diminishing throughput and increasing information content.

For example, a prescreen which accommodates up to 10⁹ samples per day can be used to quickly eliminate aborted as well as out-of-frame sequences due to insertions and deletions, e.g., by complementation assay as described above. Depending on the method of library construction, the frequency of non-functional sequence variants ranges between about 20 and 80%.

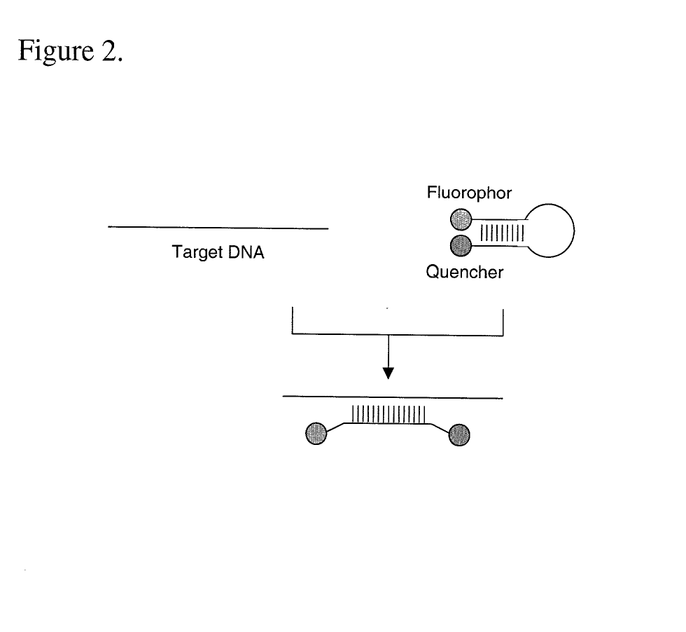

Following elimination of non-functional variants, a high throughput (HTP) functional screen is utilized to identify library members capable of reverse transcription in reaction mixtures including specified biological contaminants, e.g., blood, plasma or urine. Single bacterial colonies containing full-length, in-frame polymerase encoding sequences are identified and grown in 384-well microtiter plates, under conditions permitting induction of polymerase expression. Upon heat lysis of the host cells (e.g., E. coli) and centrifugation of cell debris, relatively pure enzyme is collected from the supernatant, and the enzyme concentration in the sample is determined. Normalized amounts of protein will be added to RT-PCR reactions in microtiter plates containing a target mRNA, the nucleotide triphosphates and various amounts of blood, plasma or urine. Detection of PCR product is performed in using fluorescent probes that hybridize to the target DNA sequence. Potential assay platforms include Taqman real-time PCR or use of molecular beacons as shown in the FIG. 2.

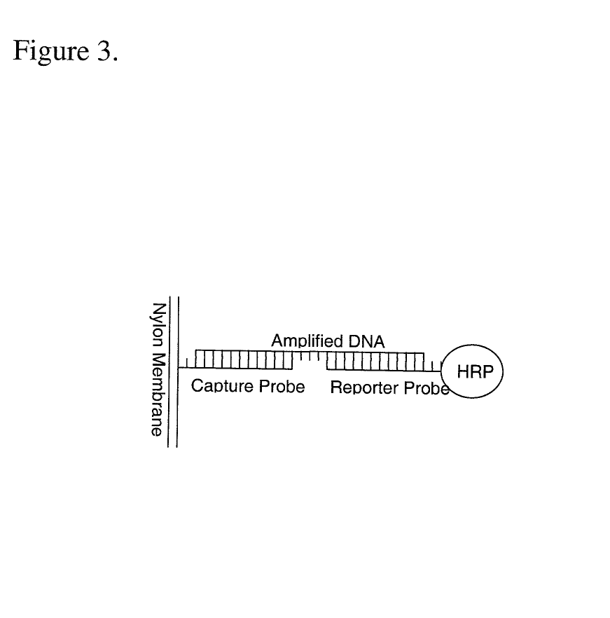

In the event that the biological fluid included in the reaction mixture results in significant quenching of the fluorescent signal (e.g., in PCR containing greater than about 20% blood), limiting the applicability of such assays, alternative approaches, e.g., involving hybridization of a linear strand of DNA to an immobilized “capture” strand of DNA can be employed. For this assay, the target DNA is deposited (“spotted”) onto a nylon (or other) filter, e.g., by a robotic apparatus, on which the capture probe is immobilized. After a short hybridization period, non-hybridizing sequences and other components of the reaction mixture that interfere with the assay are washed away. A second probe sequence, labeled with a reporter enzyme such as alkaline phosphatase or horseradish peroxidase (HRP), is then added to the membrane. After a second hybridization step, unbound secondary probe is removed, and the reporter enzyme assayed by quantitative imaging (FIG. 3).

High Throughput Screening and Hit Identification

The functional assay methods described herein and variations thereof are amenable to automation, e.g., using robotic devices, and high throughput quantitative analysis. Typically, a robot is equipped with an integrated thermocycler for HTP PCR analysis. Automated sample tracking on uniquely identifiable (e.g., bar-coded) microtiter plates is then employed. For example, multiple parallel RT-PCR evaluating properties of interest can be analyzed simultaneously, e.g., (1) with addition of plasma; (2) replacing dTTP with dUTP; (3) with addition of whole blood; and (4) with addition of urine.

Any library members that meet any of the criteria better than the best starting parent are identified as positive “hits.” Even if there no single sequence fulfills all four criteria, additional rounds of directed evolution using hits that are improved in one or more dimensions can be used to derive variants with all four traits encoded by a single nucleic acid.

Optionally, the robotic device, and associated software, can be configured to automatically identify hits from the library. The hits are automatically picked and consolidated onto one microtiter plate and used for the next round of directed evolution.

Typically, an initial determination of the amount of biological fluid that the starting polymerases can tolerate is made, and progeny polymerases are selected by incremental increases to the desired level of contaminant. For example, polymerase variants able to efficiently incorporate nucleotides in greater than about 20% blood, 25% blood, 30% blood, 35% blood, 40% blood, 45% blood, or 50% blood are incrementally selected. Similarly, polymerases active in greater than about 50% plasma, 55% plasma, 60% plasma, 65% plasma, 70% plasma, or up to about 75% plasma can be selected. As can polymerases active in about 50% urine, 55% urine, 60% urine, 65% urine, 70% urine, or up to greater than about 75% urine. In each cycle of diversification and selection an approximately 2-100 fold improvement is typically obtained, depending of the quality of library (which in turn may depend on the starting parental sequences and the diversification method utilized) and the number of variants screened. Considering the extent of biological impurity in the PCR reaction, a conservative estimate would require 3-5 rounds of evolution to produce a polymerase with the specified characteristics.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method for producing a nucleotide incorporating enzyme that incorporates a non-natural or rare nucleotide analogue, the method comprising:
(a) providing a plurality of nucleic acid segments, which nucleic acid segments encode all or part of one or more parental nucleotide incorporating enzymes or a homologue thereof;
(b) identifying at least one non-natural or rare nucleotide analogue to be incorporated by the nucleotide incorporating enzyme, which non-natural or rare nucleotide analogue is incorporated by at least one of the one or more parental nucleotide incorporating enzymes at an efficiency of less than about 10% the efficiency of a naturally occurring nucleotide;
(c) diversifying the plurality of nucleic acid segments, thereby producing a library of nucleic acids encoding nucleotide incorporating enzyme variants; and
(d) identifying at least one nucleotide incorporating enzyme variant that incorporates the non-natural or rare nucleotide analogue at least about 10% as efficiently as a naturally occurring nucleotide.

2. The method of claim 1, wherein the non-natural or rare nucleotide analogue is incorporated less efficiently than inosine by a parental nucleotide incorporating enzyme.

3. The method of claim 1, wherein the non-natural or rare nucleotide analogue is incorporated less efficiently than 7-deaza dGTP by a parental nucleotide incorporating enzyme.

4. The method of claim 1, wherein the non-natural or rare nucleotide analogue is incorporated at an efficiency of less than about 5% the efficiency of a naturally occurring nucleotide by a parental nucleotide incorporating enzyme.

5. The method of claim 1, comprising identifying a nucleotide analogue selected from the group consisting of: a nucleotide derivatized with a functional group, a nucleotide derivatized with a methyl group, a nucleotide derivatized with a nitrile group, a nucleotide comprising an unnatural base analogue, a nucleotide comprising a fluorescent label, a nucleotide comprising a ribose or deoxyribose analogue, and a nucleotide comprising an unnatural glycosidic linkage to a base.

6. The method of claim 5, wherein the nucleotide analogue is labeled at the 2′ or 3′ hydroxyl position.

7. The method of claim 6, wherein the 2′ or 3′ hydroxyl group is derivatized by direct attachment of a functional group.

8. The method of claim 7, wherein the functional group is selected from a fluoro-group, an alkoxy-group, or an amine group.

9. The method of claim 5, wherein the nucleotide analogue is labeled at a position other than the 2′ or 3′ hydroxyl position.

10. The method of claim 5, wherein the nucleotide comprising a fluorescent label comprises a fluorescent group attached via a linker or a fluorescent base analogue.

11. The method of claim 10, wherein the fluorescent group is attached by an amide linkage or an ester linkage.

12. The method of claim 5, wherein the nucleotide is derivatized by esterification or thioesterification of a phosphate.

13. The method of claim 5, wherein the nucleotide comprising a ribose or deoxyribose analogue comprises a cyclopentyl-derived sugar.

14. The method of claim 5, wherein the nucleotide comprising an unnatural glycosidic linkage to a base comprises a C-C linkage or a C-O linkage.

15. The method of claim 1, wherein the nucleic acid segments comprise RNA polynucleotides, DNA polynucleotides, or character strings representing RNA or DNA polynucleotides stored in a computer readable medium.

16. The method of claim 1, wherein the nucleic acid segments are produced by one or more of enzymatic digestion, chemical cleavage and mechanical fragmentation.

17. The method of claim 1, wherein the nucleic acid segments are artificially synthesized.

18. The method of claim 17, wherein the artificially synthesized nucleic acid segments comprise oligonucleotides.

19. The method of claim 1, wherein the plurality nucleic acid segments encode a plurality of inactive nucleotide incorporating enzymes or homologues thereof.

20. The method of claim 1, wherein the plurality of nucleic acid segments comprise a plurality of codon altered nucleic acid segments.

21. The method of claim 1, wherein the plurality nucleic acid segments comprise at least one nucleic acid segment comprising one or more intron or intein.

22. The method of claim 1, wherein the plurality of nucleic acid segments comprise sequences of two or more members of a family of nucleotide incorporating enzymes.

23. The method of claim 1, wherein the nucleic acid segments comprise members of two or more families of nucleotide incorporating enzymes.

24. The method of claim 1, wherein the one or more parental nucleotide incorporating enzyme comprises a nucleic acid polymerase, a terminal transferase, a ligase or a telomerase.

25. The method of claim 24, wherein the one or more nucleic acid polymerase comprises a DNA polymerase or an RNA polymerase.

26. The method of claim 25, wherein the one or more nucleic acid polymerase comprises a DNA polymerase or an RNA polymerase selected from the group consisting of: a DNA dependent DNA polymerase, an RNA dependent DNA polymerase, a DNA dependent RNA polymerase, or an RNA dependent RNA polymerase.

27. The method of claim 25, wherein the one or more nucleic acid polymerase comprises a thermostable DNA polymerase.

28. The method of claim 1, comprising diversifying the plurality of nucleic acid segments by at least one diversity generating procedure comprising recombining or mutating at least one nucleic acid segment.

29. The method of claim 28, comprising diversifying the plurality of nucleic acid segments by recombining the plurality of nucleic acid segments in vitro, in vivo, or in silico.

30. The method of claim 28, comprising recursively recombining the plurality of nucleic acid segments in vitro, in vivo, or in silico.

31. The method of claim 28, comprising recombining the plurality of nucleic acid segments by assembling synthetic oligonucleotides.

32. The method of claim 31, wherein the synthetic oligonucleotides are joined using only a ligase.

33. The method of claim 28, comprising diversifying the at least one nucleic acid segment by an error prone PCR.

34. The method of claim 33, further comprising recombining at least one amplified product produced by the error prone PCR.

35. The method of claim 28, comprising mutating the at least one nucleic acid segment, thereby producing one or more mutated nucleic acid segments.

36. The method of claim 35, further comprising recombining the one or more mutated nucleic acid segments.

37. The method of claim 1, wherein at least one of the nucleic acids encoding nucleotide incorporating enzyme variants further comprises a vector.

38. The method of claim 37, wherein the vector comprises a replicable vector.

39. The method of claim 1, comprising identifying the at least one nucleotide incorporating enzyme variant that incorporates the non-natural or rare nucleotide analogue by mass spectroscopy.

40. The method of claim 1, comprising identifying the at least one nucleotide incorporating enzyme variant that incorporates the non-natural or rare nucleotide analogue by one or more method selected from the group consisting of optical or fluorescent spectroscopy, radiometry, chromatography, gel electrophoresis, capillary electrophoresis, streptavidin binding, hybridization, fluorescent resonance energy transfer, fluorescent polarization, and pyrophosphate detection.

41. The method of claim 1, comprising identifying the at least one nucleotide incorporating enzyme by:
(i) transforming the library of nucleic acids into a population of host cells, growth of which host cells is dependent on the presence of at least one essential naturally occurring nucleotide in a first medium;
(ii) growing the population of transformed host cells in a second medium, which second medium lacks the at least one essential naturally occurring nucleotide of the first medium and comprises the non-natural or rare nucleotide analogue identified in step (b); and
(iii) identifying at least one surviving transformed host cell, thereby identifying a nucleic acid encoding a nucleotide incorporating enzyme variant, which nucleotide incorporating enzyme variant incorporates the non-natural or rare nucleotide analogue.

42. The method of claim 1, comprising identifying the at least one nucleotide incorporating enzyme variant in a high throughput assay format.

43. The method of claim 1, further comprising pre-screening the library of nucleic acids encoding nucleotide incorporating enzyme variants by:
(i) transforming the library of nucleic acids into a population of temperature sensitive nucleotide incorporating enzyme deficient bacterial host cells;
(ii) growing the transformed bacterial host cells at the non-permissive temperature; and
(iii) identifying one or more transformed bacterial host cells capable of growth at the non-permissive temperature, thereby identifying one or more members of the library of nucleic acids that encodes a functional nucleotide incorporating enzyme.

44. The method of claim 1, further comprising identifying at least one nucleotide incorporating enzyme variant with at least one additional desired property.

45. The method of claim 44, wherein the at least one additional desired property is selected from the group consisting of thermostability, evenness of nucleotide incorporation, efficient terminal transferase activity, low fidelity, high fidelity, processivity, strand-displacement activity, nick translation activity, exchange reaction, cation requirement, modulation of activity by cation, sulfhydral reagent requirement, shelf life, salt tolerance, organic solvent tolerance, mechanical stress tolerance, tolerance to impurities, altered pH dependence, altered dependence on buffer conditions, template composition, primer composition, and improved stability.

46. The method of claim 44, comprising identifying at least one nucleotide incorporating enzyme variant by simultaneously screening for incorporation of the non-natural or rare nucleotide analogue and at least one other desired property.

47. A method for producing a nucleotide incorporating enzyme that incorporates a non-natural or rare nucleotide analogue, the method comprising:
(a) providing a plurality of nucleic acid segments, which nucleic acid segments encode all or part of one or more parental nucleotide incorporating enzymes or a homologue thereof;
(b) identifying at least one non-natural or rare nucleotide analogue to be incorporated by the nucleotide incorporating enzyme, which non-natural or rare nucleotide analogue is incorporated by at least one of the one or more parental nucleotide incorporating enzymes at an efficiency of less than about 10% the efficiency of a naturally occurring nucleotide;
(c) diversifying the plurality of nucleic acid segments, thereby producing a library of nucleic acids encoding nucleotide incorporating enzyme variants; and
(d) identifying at least one nucleotide incorporating enzyme variant that incorporates the non-natural or rare nucleotide analogue at least about 10 fold more efficiently than at least one of the one or more parental nucleotide incorporating enzyme.

48. The method of claim 47, wherein the non-natural or rare nucleotide analogue is incorporated at an efficiency of less than about 5% the efficiency of a naturally occurring nucleotide.

49. The method of claim 47, wherein the non-natural or rare nucleotide analogue is incorporated at an efficiency of less than about 1% the efficiency of a naturally occurring nucleotide.

50. The method of claim 47, wherein the non-natural or rare nucleotide analogue is incorporated at an efficiency of less than about 0.5% the efficiency of a naturally occurring nucleotide.

51. The method of claim 47, wherein the non-natural or rare nucleotide analogue is incorporated at an efficiency of less than about 0.1% the efficiency of a naturally occurring nucleotide.

52. The method of claim 47, wherein the non-natural or rare nucleotide analogue is incorporated at an efficiency of less than about 0.05% the efficiency of a naturally occurring nucleotide.

53. The method of claim 47, wherein the non-natural or rare nucleotide analogue is incorporated at an efficiency of less than about 0.01% the efficiency of a naturally occurring nucleotide.

54. The method of claim 47, wherein the at least one nucleotide incorporating enzyme variant identified in step (d) incorporates the non-natural or rare nucleotide analogue at least about 20 fold more efficiently than at least one of the one or more parental nucleotide incorporating enzyme.

55. The method of claim 47, wherein the at least one nucleotide incorporating enzyme variant identified in step (d) incorporates the non-natural or rare nucleotide analogue at least about 50 fold more efficiently than at least one of the one or more parental nucleotide incorporating enzyme.

56. The method of claim 47, wherein the at least one nucleotide incorporating enzyme variant identified in step (d) incorporates the non-natural or rare nucleotide analogue at least about 100 fold more efficiently than at least one of the one or more parental nucleotide incorporating enzyme.

57. The method of claim 1 or 47, further comprising extending a plurality of nucleic acid segments annealed to a single stranded template using the nucleotide incorporating enzyme variant identified in step (d).

58. The method of claim 1 or 47, further comprising performing at least one PCR using the nucleotide incorporating enzyme variant identified in step (d).

59. The method of claim 1 or 47, further comprising performing at least one sequencing reaction using the nucleotide incorporating enzyme variant identified in step (d).

60. A method for producing a nucleotide incorporating enzyme with increased tolerance to biological impurities, the method comprising:
(a) providing a plurality of nucleic acid segments, which nucleic acid segments encode all or part of one or more parental nucleotide incorporating enzymes or a homologue thereof;
(b) diversifying the plurality of nucleic acid segments, thereby producing a library of nucleic acids encoding nucleotide incorporating enzyme variants; and
(c) identifying at least one nucleotide incorporating enzyme variant that efficiently polymerizes a polynucleotide in a template dependent manner in the presence of a biological impurity found in blood, plasma or urine.

61. The method of claim 60, wherein the nucleotide incorporating enzyme is a DNA polymerase.

62. The method of claim 60, wherein the nucleotide incorporating enzyme is an RNA dependent DNA polymerase.

63. The method of claim 60, wherein the nucleotide incorporating enzyme is a reverse transcriptase.

64. The method of claim 60, comprising providing a plurality of nucleic acid segments comprising a parental polynucleotide incorporating enzyme from at least one thermophilic organism.

65. The method of claim 60, comprising providing a plurality of nucleic acid segments comprising at least one bacterial DNA Polymerase I.

66. The method of claim 60, wherein the at least one bacterial DNA Polymerase I is a Thermus DNA Polymerase I.

67. The method of claim 60, wherein the at least one nucleotide incorporating enzyme identified in step (c) is identified in a high throughput assay.

68. The method of claim 60, wherein the at least one nucleotide incorporating enzyme identified in step (c) comprises a thermostable enzyme.

69. The method of claim 60, wherein the at least one nucleotide incorporating enzyme identified in step (c) comprises an enzyme that is capable of incorporating dUTP.

70. The method of claim 60, wherein the at least one nucleotide incorporating enzyme identified in step (c) comprises an enzyme that is incorporates dUTP at least as efficiently as a nucleotide incorporating enzyme selected from the group consisting of T aquaticus DNA polymerase (Taq) and T thermophilus DNA polymerase (Tth).

71. The method of claim 60, wherein the at least one nucleotide incorporating enzyme identified in step (c) is active in a reaction mixture comprising at least about 20% blood.

72. The method of claim 60, wherein the at least one nucleotide incorporating enzyme identified in step (c) is active in a reaction mixture comprising at least about 50% plasma.

73. The method of claim 60, wherein the at least one nucleotide incorporating enzyme identified in step (c) is active in a reaction mixture comprising at least about 50% urine.

74. A nucleotide incorporating enzyme variant produced by the method of claim 1, 47 or 60.

75. The nucleotide incorporating enzyme variant of claim 74, which nucleotide incorporating enzyme variant incorporates the non-natural or rare nucleotide analogue at least about 10% as efficiently as a naturally occurring nucleotide.

76. The nucleotide incorporating enzyme variant of claim 74, which nucleotide incorporating enzyme variant incorporates the non-natural or rare nucleotide analogue at least about 10 fold more efficiently than at least one of the one or more parental nucleotide incorporating enzymes.

77. The nucleotide incorporating enzyme variant of claim 74, which variant incorporates nucleotides or nucleotide analogues with low fidelity.

78. The nucleotide incorporating enzyme variant of claim 74, which variant incorporates nucleotides or nucleotide analogues with high fidelity.

79. A kit comprising the nucleotide incorporating enzyme variant of claim 74, and one or more of a container, a packaging material, and a natural nucleotide or non-natural or rare nucleotide analogue.

80. A kit comprising the nucleotide incorporating enzyme variant of claim 75, and one or more of a container, a packaging material, and a non-natural or rare nucleotide analogue, which non-natural or rare nucleotide analogue is incorporated by a parental nucleotide incorporating enzyme at an efficiency of less than 10% the efficiency of a naturally occurring nucleotide, and which non-natural or rare nucleotide analogue is incorporated by the nucleotide incorporating enzyme variant at least 10% as efficiently as a naturally occurring nucleotide.

81. A kit comprising the nucleotide incorporating enzyme variant of claim 76, and one or more of a container, a packaging material, and a non-natural or rare nucleotide analogue, which non-natural or rare nucleotide analogue is incorporated by a parental nucleotide incorporating enzyme at an efficiency of less than 10% the efficiency of a naturally occurring nucleotide, and which non-natural or rare nucleotide analogue is incorporated by the nucleotide incorporating enzyme variant at least 10 fold more efficiently than it is incorporated by at least one of the one or more parental nucleotide incorporating enzymes.

82. An integrated system comprising a non-natural nucleotide analogue, a nucleotide incorporating enzyme variant produced by the method of claim 1, 47, or 74 and a detector.

83. The integrated system of claim 82, further comprising one or more of a user input device, a data processing device, a data output device, and a robotic controller.

84. The method of claim 1, wherein the nucleic acid segments are oligonucleotides, and wherein the diversifying step (c) comprises:
i) generating a plurality of partially duplexed oligonucleotides by hybridizing the oligonucleotides from step (a), said duplexed oligonucleotides having overhangs of unhybridized regions;
ii) assembling the plurality of partially duplexed oligonucleotides by hybridizing the overhangs of two or more partially duplexed oligonucleotides together; and,
iii) ligating the assembled oligonucleotides to produce a library of recombinant nucleic acids, optionally in the presence of a polymerase.

85. The method of claim 60, wherein the nucleic acid segments are oligonucleotides, and wherein the diversifying step (b) comprises:
i) generating a plurality of partially duplexed oligonucleotides by hybridizing the oligonucleotides from step (a), said duplexed oligonucleotides having overhangs of unhybridized regions;
ii) assembling the plurality of partially duplexed oligonucleotides by hybridizing the overhangs of two or more partially duplexed oligonucleotides together; and,
iii) ligating the assembled oligonucleotides to produce a library of recombinant nucleic acids, optionally in the presence of a polymerase.

86. A method for identifying a nucleotide incorporating enzyme with having a desired property, the method comprising:
a) providing a plurality of partially duplexed oligonucleotides having overhangs of unhybridized regions, which oligonucleotides comprise a subsequence of a nucleic acid encoding a nucleotide incorporating enzyme;
b) assembling the plurality of partially duplexed oligonucleotides by hybridizing the overhangs of two or more partially duplexed oligonucleotides together;
c) ligating the assembled oligonucleotides to produce a library of recombinant nucleic acids, optionally in the presence of a polymerase;
d) expressing the recombinant nucleic acids to generate a library of nucleotide incorporating enzyme variants; and,
e) screening the library of nucleotide incorporating enzyme variants for one or more desired property.

87. The method of claim 86, wherein the one or more desired property is selected from the group consisting of: incorporation of rare or non-natural nucleotides, thermostability, evenness of nucleotide incorporation, efficient terminal transferase activity, low fidelity, high fidelity, processivity, strand-displacement activity, nick translation activity, exchange reaction, cation requirement, modulation of activity by cation, sulfhydral reagent requirement, shelf life, salt tolerance, organic solvent tolerance, mechanical stress tolerance, tolerance to impurities, altered pH dependence, altered dependence on buffer conditions, template composition, primer composition, and improved stability.
